(19) United States
(12) Patent Application Publication
(10) Pub. No.: US 2015/0183823 A1
(43) Pub. Date: Jul. 2, 2015

(54) COSMETIC OR PHARMACEUTICAL COMPOSITIONS INCLUDING TRIPEPTIDES CAPABLE OF STIMULATING CYCLIC ADENOSINE MONOPHOSPHATE SYNTHESIS AND THEIR USE IN THE TREATMENT AND/OR CARE OF THE SKIN AND/OR HAIR

(71) Applicant: Lipotec, S.A., Gava (ES)

(72) Inventors: Núria GARCÍA SANZ, El Campello (ES); Wim VAN DEN NEST, Vilanova i la Geltru (ES); Cristina CARREÑO SERRAÏMA, Barcelona (ES); Antonio Vicente FERRER MONTIEL, Alicante (ES); Juan CEBRIÁN PUCHE, Barcelona (ES); Núria ALMIÑANA DOMENECH, Barcelona (ES)

(73) Assignee: LIPOTEC, S.A.

(21) Appl. No.: 14/643,434

(22) Filed: Mar. 10, 2015

Related U.S. Application Data
(62) Division of application No. 13/257,807, filed on Sep. 20, 2011.
(60) Provisional application No. 61/170,891, filed on Apr. 20, 2009.

(30) Foreign Application Priority Data
Apr. 17, 2009 (ES) 200901012

(51) Int. Cl. C07K 5/08; C07K 1/04; A61Q 1/06; A61Q 17/04; A61Q 1/02; C07K 1/02; A61Q 19/00

(52) U.S. Cl. CPC C07K 5/08; C07K 1/02; C07K 1/04; A61Q 19/00; A61Q 17/04; A61Q 1/02; A61Q 1/06

(57) ABSTRACT

Cosmetic or pharmaceutical compositions and methods employ peptides of general formula (I):

R₁-AA₁-AA₂-AA₃-R₂, (I)
where AA₁ is selected from -Tyr- and -Phe-, AA₂ is -Tyr-, and AA₃ is selected from -Nle- and -Met-,
its stereoisomers, mixtures thereof and/or their cosmetically or pharmaceutically acceptable salts, in the treatment and/or care of conditions, disorders and/or diseases of the skin and/or hair by stimulating cyclic adenosine monophosphate synthesis (cAMP).

### CROSS-REFERENCE TO RELATED APPLICATIONS

This application is a divisional of U.S. application Ser. No. 13/257,807, filed Sep. 20, 2011, which is the U.S. national phase of PCT Appln. No. PCT/EP2010/002348 filed Apr. 16, 2010 which claims priority to Spanish application 200901012 filed Apr. 17, 2009 and claims the benefit of U.S. provisional application Ser. No. 61/170,891 filed Apr. 20, 2009, the disclosures of which are incorporated in their entireties by reference herein.

### FIELD OF THE INVENTION

This invention relates to peptides capable of stimulating cyclic adenosine monophosphate synthesis (cAMP) in the skin and/or hair and cosmetic or pharmaceutical compositions containing these peptides used in the treatment and/or care of the skin and/or hair, preferably for the treatment and/or care of those conditions, disorders and/or diseases of the skin and/or hair which require stimulation of cAMP synthesis.

### BACKGROUND OF THE INVENTION

The color of the skin and the hair is due principally to a specialized dendritic cell population present in the epidermis, the melanocytes. This cell type is located in hair follicles associated to these melanocytes, in the basal lamina of the interfollicular epidermis and in the nervous system. The mature melanocytes develop ramifications which are in contact with the keratinocytes, to which they transfer vesicles containing the pigment that they synthesize: melanin. One of the functions of melanin is to protect the cell's genetic material from lesions or mutations induced by the ultraviolet radiation (UV) present in sunlight, since it absorbs until 90% of UV radiation. Melanin also protects the skin from the effect of aging accelerated by UV radiation, known as photoaging. The terms “aging” and “photoaging” of the skin relate to visible changes in the aspect of the skin such as wrinkles, fine lines, roughness, expression lines, stretch marks, discontinuities, furrows, flaccidity, sagging of the skin such as sagging cheeks, loss of resilience, loss of firmness, elastosis, keratosis, and loss of smoothness.

Several chemical compounds with their own characteristics are grouped under the melanin name. Eumelanin is black, whilst pheomelanin adopts a lighter color, which is between a reddish color and yellow. Skin and hair tonality is determined by the proportion of one or another type of pigment. These pigments accumulate in the melanosomes of the melanocyte cytoplasm and are transported by the melanosomes to the dendrites where they are injected into the cytoplasm of the basal cells. Thus a homogenous distribution of melanin is produced in the basal layer of the epidermis giving the skin a uniform pigmentation [Hearing V. J. (1999) “Biochemical control of melanogenesis and melanosomal organization” J. Invest. Dermatol. 4:24-28]. In the same way, hair color depends on the quantity and quality of the melanin located in the cortex of the hair shaft. This melanin is produced by the melanocytes located in the base of the root and depends on hereditary, hormonal or nutritional factors among others. Over the years the quantity of melanin in hair decreases due to a reduction in the activity of the melanocytes, explaining the graying of hairs. There is a third type of melanin: neuromelanin, located in the central nervous system and responsible for the color of the substantia nigra and the locus coeruleus.

The melanin pigmentation of skin can be divided into several causal components: 1) cutaneous melanin generated in accordance with genetic programs in the absence of exposure to ultraviolet rays (constitutive skin color) and 2) the reactions of immediate and delayed tanning induced by the direct exposure of skin to UV radiation (facultative skin color). The changes in facultative color are a consequence of the interaction between sunlight, hormones and the ability to tan, this depending on the genetic constitution of each individual.

UV radiation stimulates melanogenesis in different ways. UV radiation causes modifications to the membrane phospholipids, a fact which gives rise to the activation of phospholipase C. Phospholipase C causes the freeing of diacylglycerol, capable of activating the protein kinase C (PKC), and this activates the tyrosinase enzyme [Nishizuka Y. (1986) “Studies and perspectives of protein kinase C” Science 233:305-312; Park H. Y., Perez J. M., Laursen R., Hara M. and Gilchrest B. A. (1999) “Protein kinase C-beta activates tyrosinase by phosphorylating serine residues in its cytoplasmic domain” J. Biol. Chem. 274:16470-16478]. UV radiation also acts on the production of the nitric oxide and cyclic guanosine monophosphate (cGMP) messengers [Roméro-Graillet C., Aberdam E., Biagoli N., Massabni W, Ortonne J. P. and Ballotti R. (1996) “Ultraviolet B radiation acts through the nitric oxide and cGMP signal transduction pathway to stimulate melanogenesis in human melanocytes” J. Biol. Chem. 271:28052-28056]. Another effect of UV radiation is stimulation of the production of proopiomelanocortin peptides as the melanocyte-stimulating hormone (α-MSH), and the adrenocorticotropic hormone (ACTH) in keratinocytes [Hunt G., Donatien P. D., Lunec J., Todd C., Kyne S, and Thody A. J. (1994) “Cultured human melanocytes respond to MSH peptides and ACTH” Pigment Cell Res. 7:217-221]; additionally, UV radiation negatively regulates expression of neprilysin, a peptidase which cuts and inactivates α-MSH and ACTH [Aberdam E., Auberger P., Ortonne J. P. and Ballotti R. (2000) “Neprilysin, a novel target for ultraviolet B regulation of melanogenesis via melanocortins” J. Invest. Dermatol. 115:381-387].

α-MSH binds to the human melanocortin-1 receptor (MC1Rα), bound to the Gα protein, which activates adenylyl cyclase (AC) and this leads to an increase in intracellular cAMP. The increase in cAMP causes an increase in the expression of the catalytic subunit of protein kinase A (PKA), which can phosphorylate and activate the cAMP response element-binding protein (CREB). The interaction of the CREB transcription factor with the cAMP response element (CRE) sequence of the promoter of the microphthalmia-associated transcription factor (MITF) gene stimulates its expression. MITF is a transcription factor which modulates the expression of several key enzymes for melanin synthesis, such as tyrosinase, dopachrome tautomerase (DCT) and the tyrosinase-related protein 1 (TRP-1) [Bertolotto C., Bille K., Ortonne J. P. and Ballotti R. (1996) “Regulation of tyrosinase gene expression by cAMP in B16 melanoma cells involves two CATGTG motifs surrounding the TATA box: implication of the microphthalmia gene product” J. Cell Biol. 134:747-55; Bertolotto C., Buscà R., Abbe P., Bille K., Aberdam E., Ortonne J. P. and Ballotti R. (1998) “Different cis-acting elements are involved in the regulation of TRP1 and TRP2 promoter activities by cyclic AMP: pivotal role of M boxes (GTCATGTGCT) and of microphthalmia” Mol. Cell. Biol. 18:694-702; Bertolotto C., Abbe P., Hemesath T. J., Bille K., Fisher D. E., Ortonne J. P. and Ballotti R. (1998) “Microphthalmia gene product as a signal transducer in cAMP-induced differentiation of melanocytes” J. Cell Biol. 142:827-35]. Tyrosinase, the only one which is essential for melanogenesis, catalyzes two initial restricting reactions of the process: tyrosine hydroxylation which leads to 3,4-dihydroxyphenylalanine (DOPA) and the oxidation of DOPA leads to dopaquinone. DCT, in turn, isomerizes dopaquinone to 5,6-dihydroxyindole-2-carboxylic acid, and this is polymerized to melanin [Chakraborty A. K., Platt J. T., Kim K. K., Kwon B. S., Bennett D. C. and Pawelek J. M. (1996) “Polymerization of 5,6-dihydroxyindole-2-carboxylic acid to melanin by the pmel 17/silver locus protein” Eur. Biochem. 236:180-188].

It is known that during the life of a person apparent changes in the coloring of his/her skin develop and thus, for example, marks on the skin of the face, chest and hands of elderly people appear which are clear signs of aging [Piérard G. E., Piérard-Franchimont C., Laso Dosal F., Ben Mosbah T., Arrese Estrada J., Rurangirwa A., Dowlati A. and Vardar M. (1991) “Pigmentary changes in skin senescence” J. Appl. Cosmetol. 9:57-63]. Furthermore, the continuous overexposure to UV radiation does not just cause accelerated aging of the skin, known as photoaging, which is characterized by the appearance of signs of skin aging at a much earlier age, among them the appearance of marks in those areas of the skin overexposed to UV radiation [Stefanaki C., Stratigos A. and Katsambas A. (2005) “Topical retinoids in the treatment of photoaging” J. Cosmet. Dermatol. 4:130-134], but which can also give rise to the formation of hyperpigmented cancerous lesions or melanomas [Dooley T. P. (1994) “Recent advances in cutaneous melanoma oncogenesis research” Onco. Res. 6:1-9].

It frequently occurs that in an area of an person's skin the density of melanin within the melanocytes is greater than in the surrounding areas and as a consequence the color of the affected area on that person is darker than the rest. These areas are known as areas of hyperpigmentation. Among the causes of hyperpigmentation are hormonal changes, melasma, lentigo, piebaldism, Addison's disease, hypersensitivity to ultraviolet radiation due to agents which favor the action of radiation (phototoxics), or hyperpigmentation as a consequence of an inflammatory lesion. The marks associated with acne, eczema, scars or hair removal belong to this last type of hyperpigmentation and are marks that can even last several years.

It is also possible for a person's skin areas which have lower melanin densities than that in surrounding areas. A skin disease which presents this type of hypopigmentary marks is vitiligo [Benmaman O. and Sanchez J. L. (1988) “Treatment and camouflaging of pigmentary disorders” Clin. Dermatol. 6:50-61; Schallreuter K. U. (1997) “Epidermal adrenergic signal transduction as part of the neuronal network in the human epidermis” J. Invest. Dermatol. 2:37-40].

The attenuation of the irregularities of pigmentation are either due to aging and/or photoaging, to hormonal disorders or to post-inflammatory processes and, particularly, the re-establishment of the pigmentation in the areas affected by vitiligo with topical applications is, therefore, of interest to the cosmetic and pharmaceutical sector.

For several decades, tanned skin has been associated with a healthy appearance by associating the skin color achieved as a consequence of carrying out outdoor physical and/or leisure activities, therefore having tanned skin is considered desirable for a great number of people. However, different studies suggest that the individuals who have skin which does not tan easily and tends to burn when exposed to UV radiation are at greater risk of developing cutaneous melanomas and non-malignant skin tumors [Stenback F. (1978) “Life history and histopathology of ultraviolet light-induced skin tumors” Natl. Cancer Inst. Monogr. 50:57-70; Kricker A., Armstrong B. K., English D. R. and Heenan P. J. (1995) “Does intermittent sun exposure cause basal cell carcinoma? A case-control study in Western Australia” Int. J. Cancer 60:489-494], therefore the medical sector has strived to heighten awareness of the risks of prolonged exposure to UV radiation. Individuals classified in the Fitzpatrick scale as phototypes I and II burn easily [Fitzpatrick T. B. (1988) “The validity and practicality of sun-reactive skin types I through VI” Arch. Dermatol. 124:869-871], presenting a greater risk of developing skin cancer [Sober A. J., Lew R. A., Koh H. K. and Barnhill R. L. (1991) “Epidemiology of cutaneous melanoma. An update” Dermatol. Clin. 9:617-629; Palmer J. S., Duffy D. L., Box N. F., Aitken J. F., O'Gorman L. E., Green A. C., Hayward N. K., Martin N. G. and Sturm R. A. (2000) “Melanocortin-1 receptor polymorphisms and risk of melanoma: is the association explained solely by pigmentation phenotype?” Am. Hum. Genet. 66:176-186; Box N. F., Duffy D. L., Irving R. E., Russell A., Chen W., Griffyths L. R. Parsons P. G., Green A. C. and Sturm R. A. (2001) “Melanocortin-1 receptor genotype is a risk factor for basal and squamous cell carcinoma” J. Invest. Dermatol. 116:224-229].

The stimulation of melanin synthesis without the risks associated with the damage caused by UV radiation arises, particularly in populations with low levels of pigmentation, from a medical point of view, as a strategy of desired photoprotection for the reduction of the incidence of skin cancer in the world [Armstrong B. K. and Kricker A. (1994) “Cutaneous melanoma” Cancer Survey 19/20:219-240]. Furthermore, stimulation of melanin synthesis without the risks associated with the damage caused by UV radiation is desirable from a cosmetic point of view to achieve a quick, intense and lasting tan in a risk-free way.

In the same way, within the beauty standards established in the majority of countries and races, white hair, known as gray hairs, is not desirable since it is associated with old age. During aging, the majority of people develop a gradual depigmentation of the hair, and melanogenesis can even be completed inhibited in the melanocytes associated with hair follicles. Likewise, gray hairs often appear on people subjected to stressful situations, people with vitamin B deficiency anemia, or in people with thyroid disorders. Therefore, there is an interest in the availability of curative or preventative treatments capable of maintaining the process of hair pigmentation and of stimulating melanogenesis and pigmentation of hairs which have a tendency to turn gray.

The interest in achieving tanned skin, for both aesthetic and therapeutic purposes, as well as maintaining hair with its natural level of pigmentation is reflected in the effort carried out both by the cosmetic and the pharmaceutical industry in developing products capable of stimulating melanogenesis and that are capable of accelerating, intensifying and/or prolonging the skin's tan.

Exposure to UV radiation, whether from sunlight or UV fluorescent lamps, does not just accelerate skin aging, a process known as photoaging, but also results in an increase in the incidence of skin cancer. There is, therefore, the need for cosmetic or pharmaceutical agents, compositions and methods to give the skin a tanned look with the minimum time of exposure to UV radiation, and, therefore, with a lower risk of damage induced by UV radiation. Likewise, there is an interest in having cosmetic or pharmaceutical agents, compositions and methods to accelerate, intensify and prolong the skin's tan with the aim of providing the skin with a faster and longer lasting protection against UV radiation.

A strategy widely used in the cosmetic sector to give the skin a tanned look is the use of make-up. However, the use of make-up does not afford a lasting color and requires a long time to apply. Furthermore, make-up has the drawback of dirtying clothes which come into contact with the skin, particularly around the neck area. A more permanent type of bronzing is that offered by the use of dihydroxyacetone (DHA) and analogues or erythrulose. Tanning of the skin by these compounds is independent from that produced by exposure to UV radiation and is caused by the Maillard reaction between them and the skin's amino acids and amino groups in keratin [Bobin M. F., Martini M. C. and Cotte J. (1984) “Effects of Color Adjuvants on the Tanning Effect of Dihydroxyacetone” J. Soc. Cosmet. Chem. 35:265-272]. The resulting color is usually too orangish and unnatural; furthermore, this tan has none of the beneficial effects of the increase in cutaneous melanin, such as the protective effect on DNA against UV radiation. In the same way, the cosmetic sector has used products containing pigments such as beta-carotene and canthaxanthin; however, they also give an unnatural color and offer little protection against UV radiation compared with a natural tan. Another related strategy is the administration of the melanin itself in a composition which contains it. The problem with this strategy is the insolubility of the actual polymer or the difficulties of achieving a uniform level of polymerization of melanin. Joint administrations of pigments and a vehicle to bind them are also described, such as those described in patent U.S. Pat. No. 7,081,442, in which a pigment and peptides are combined to achieve the darkening of the skin; or in patent U.S. Pat. No. 7,220,405, in which peptides are used to bind a pigment present in the same formulation to skin and hair. Formulations are also described which combine DHA with other ingredients to induce the darkening of the skin, such as those described in documents U.S. Pat. No. 5,503,824 or GB2413763.

An approach to achieve a more natural tan is the induction of melanin synthesis, which permits the same effects to be achieved as in tanning through to exposure to the sun without submitting the skin to the risks of ultraviolet radiation. It is known in the prior art that the induction of melanin synthesis through the application of psoralens, which are photosensitizing agents and, therefore, increases the quantity of melanin when combined with exposure to UV radiation. Psoralens do not darken the skin without exposure to UV; therefore they should be administered with precaution to minimize the risk of skin cancer. The administration of psoralens, together with medical grade UV lamps, is an accepted treatment for vitiligo and psoriasis, but are not recommendable for people just looking for a tan.

The administration of tyrosine and its derivatives, such as acetyl tyrosine or oleoyl tyrosine, is widely known in the prior art as pro-melanogenic agents, since they act as substrates of the enzyme tyrosine increasing their activity. An induction of melanin synthesis through the administration of compounds which increase cAMP levels can also be achieved, such as glycyrrhizin, forskolin, α-MSH and derived peptides, peptides derived from the melanocortin receptor or, xanthine and derivatives such as isobutylmethylxanthine (IBMX) or theophylline. The pharmaceutical industry has developed a α-MSH analogue known as afamelanotide or melanotan-I (Nle⁴-D-Phe⁷-α-MSH) with the aim of fighting melanomas through stimulation of melanogenesis minimizing exposure to UV radiation. Afamelanotide is currently found in clinical trials (Bametson R. S. C, Ooi T. K. T., Zhuang L., Halliday G. M., Reid C. M., Walker P. C., Humphrey S. M. and Kleinig M. J. (2006) “[Nle⁴-D-Phe⁷]-α-Melanocyte-Stimulating Hormone Significantly Increased Pigmentation and Decreased UV Damage in Fair-Skinned Caucasian Volunteers” J. Invest. Dermatol. 126:1869-1878]. The cosmetic field has also used cAMP synthesis-promoting agents to induce both melanin and forskolin synthesis. However, forskolin has its disadvantages due to its low solubility in aqueous solutions (Lal B., Gangopadhyay A. K., Gidwani R. M., Fernandes M., Rajagopalan R. and Ghate A. V. (1998) “In search of novel water soluble forskolin analogues for positive inotropic activity” Bioorg. Med. Chem. 6:2075-2083], which undoubtedly causes difficulties for the formulation at an industrial scale of the compositions which contain it. Different patents applied in the cosmetic field which describe compositions which act on cAMP levels are found in the prior art, such as patent FR2,691,465 which claims the use of peptides derived from α-MSH to achieve self-tanning effects; these peptides can be attached to polysaccharides produced by bacteria of the genus Klebsiella.

There is still a need to identify new agents capable of stimulating cAMP synthesis in the skin and/or hair and therefore capable of accelerating, intensifying and/or prolonging the skin's tan for its co-administration with the existing agents with the aim of achieving better results in the pigmentation of the skin and/or hair, and in particular, to intensify the skin's tan, minimizing the exposure time to UV radiation.

It is known in the prior art that cAMP is a secondary messenger involved in the process of fat accumulation in the adipocytes. The net fat storage or elimination in the adipocyte depends on the balance between the uptake of triglycerides in the diet which travel in the chylomicrons in the blood and the break-down of the triglycerides stored in the adipocytes with the resulting elimination of free fatty acids for their subsequent use as a source of energy. This break-down of triglycerides in the adipocyte, known as lipolysis, is caused when a hormone-sensitive lipase (HSL) is activated. The activation of the HSL requires the phosphorylation by cAMP dependant on a protein kinase. Therefore, cAMP is a limiting factor for lipolysis. The net quantity of cAMP is the result of the balance between its enzymatic synthesis from adenosine triphosphate (ATP) by adenylate cyclise, and its break-down by phosphodiesterases. The majority of treatments for cellulite focus on lipolysis as a principal means of action. The use of agents stimulating cAMP synthesis such as lipolytic agents is known in the prior art [Allen D. O., Ahmed B. and Naseer K. (1986) “Relationships between cyclic AMP levels and lipolysis in fat cells after isoproterenol and forskolin stimulation” J. Pharmacol. Exp. Ther. 238:659-664] and the cosmetic sector has developed compositions which contain these types of agents for the treatment and/or care of conditions, disorders and/or diseases which require a stimulation of lipolysis such as, for example, cellulite [U.S. Pat. No. 7,476,392; U.S. Pat. No. 4,525,359]. These compositions basically contain forskolin and derivatives and, therefore, their production at an industrial scale poses the same problems derived from the low solubility of forskolin.

There is also, therefore, a need to identify new agents capable of stimulating cAMP synthesis in the skin for its co-administration with the existing agents with the aim of stimulating lipolysis and achieve better results in the treatment and/or care of cellulite.

In this invention peptides capable of increasing cAMP synthesis are described and, which are therefore capable of stimulating melanin synthesis in the skin and/or hair and accelerating, intensifying and/or prolonging the skin's tan, as well as stimulating lipolysis and treating and/or caring for cellulite. These peptides do not stem from the α-MSH sequence or from the melanocortin receptor, therefore a person skilled in the art could not deduce the efficiency of these peptides as promoters of cAMP synthesis.

### DETAILED DESCRIPTION OF THE INVENTION

This invention provides a solution to the above-mentioned problem. Surprisingly, the applicant of this invention has found that synthetic peptides not stemming from the α-MSH sequence or the melanocortin receptor exhibit a significant efficiency in the induction of cAMP synthesis and therefore are capable of stimulating melanin synthesis in the skin and/or hair and stimulating lipolysis. These peptides are used in the treatment and/or care of the skin and/or hair, preferably for the treatment and/or care of those skin and/or hair conditions, disorders and/or diseases which require a stimulation of cAMP synthesis.

### DEFINITIONS

In order to facilitate the comprehension of this invention, the meanings of some terms and expressions as they are used within the context of the invention are included.

Within the context of this invention “skin” is understood to be the layers which comprise it from the outermost layer or stratum corneum to the lowermost layer or hypodermis, both inclusive. These layers are comprised by different types of cells such as keratinocytes, fibroblasts, melanocytes and/or adipocytes among others.

In this description the abbreviations used for the amino acids follow the IUPAC-IUB Joint Commission on Biochemical Nomenclature rules outlined in Eur. J. Biochem. (1984) 138:9-37 and in J. Biol. Chem. (1989) 264:633-673.

Thus, for example, Nle represents NH₂—CH[(CH₂)₃CH₃]—COOH, Nle- represents NH₂—CH[(CH₂)₃CH₃]—CO—, -Nle represents —NH—CH[(CH₂)₃CH₃]—COOH and -Nle- represents —NH—CH[(CH₂)₃CH₃]—CO—. Therefore, the dash, which represents the peptide bond, eliminates the OH of the 1-carboxyl group of the amino acid (represented here in the non-ionized conventional form) when located at the right of the symbol, and eliminates the H of the 2-amino group of the amino acid when located at the left of the symbol; both modifications can be applied to the same symbol (see Table 1).

TABLE 1
Amino acid structures and their three letter
nomenclature code.

| Symbol | Remainder |
|---|---|
| -Nle- | 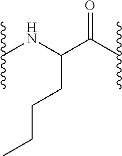 [SMILES: *NC(CCCC)C(*)=O] |
| -Phe- | 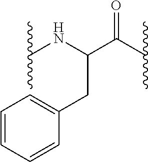 [SMILES: *NC(Cc1ccccc1)C(*)=O] |
| -Met- | 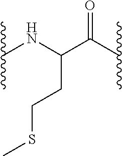 [SMILES: *NC(CCSC)C(*)=O] |
| -Tyr- | 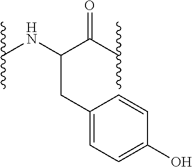 [SMILES: *NC(Cc1ccc(O)cc1)C(*)=O] |

The abbreviation “Ac-” is used in this description to name the acetyl group (CH₃—CO—) and the abbreviation “Palm-” is used to name the palmitoyl group (CH₃—(CH₂)₁₄—CO—)

The term “non-cyclic aliphatic group” is used in this invention to cover, for example and not restricted to, linear or branched alkyl, alkenyl and alkynyl groups.

The term “alkyl group” relates to a saturated, linear or branched group, which has between 1 and 24, preferably between 1 and 16, more preferably between 1 and 14, even more preferably between 1 and 12, and even more preferably still between 1, 2, 3, 4, 5 or 6 carbon atoms and which is bound to the rest of the molecule by a single bond, including, for example and not restricted to, methyl, ethyl, isopropyl, isobutyl, tert-butyl, heptyl, octyl, decyl, dodecyl, lauryl, hexadecyl, amyl, 2-ethylhexyl, 2-methylbutyl, 5-methylhexyl and similar.

The term “alkenyl group” refers to a linear or branched group which has between 2 and 24, preferably between 2 and 16, more preferably between 2 and 14, even more preferably between 2 and 12, even more preferably still 2, 3, 4, 5 or 6 carbon atoms, with one or more carbon-carbon double bonds, preferably with 1, 2 or 3 carbon-carbon double bonds, conjugated or unconjugated, which is bound to the rest of the molecule through a single bond, including, for example and not restricted to, the vinyl, oleyl, linoleyl and similar groups.

The term “alkynyl group” refers to a linear or branched group which has between 2 and 24, preferably between 2 and 16, more preferably between 2 and 14, even more preferably between 2 and 12, even more preferably still 2, 3, 4, 5 or 6 carbon atoms, with one or more carbon-carbon triple bonds, preferably with 1, 2 or 3 carbon-carbon triple bonds, conjugated or unconjugated, which is bound to the rest of the molecule through a single bond, including, for example and not restricted to, the ethinyl group, 1-propinyl, 2-propinyl, 1-butinyl, 2-butinyl, 3-butinyl, pentinyl, such as 1-pentinyl and similar groups.

The term “alicyclic group” is used in this invention to cover, for example and not restricted to, cycloalkyl or cycloalkenyl or cycloalkynyl groups.

The term “cycloalkyl” relates to a saturated mono- or polycyclic aliphatic group which has between 3 and 24, preferably between 3 and 16, more preferably between 3 and 14, even more preferably between 3 and 12, even more preferably still 3, 4, 5 or 6 carbon atoms and which is bound to the rest of the molecule through a single bond, including, for example and not limited to, cyclopropyl, cyclobutyl, cyclopentyl, cyclohexyl, cycloheptyl, methyl cyclohexyl, dimethyl cyclohexyl, octahydroindene, decahydronaphthalene, dodecahydro-phenalene and similar.

The term “cycloalkenyl” relates to a non-aromatic mono- or polycyclic aliphatic group which has between 5 and 24, preferably between 5 and 16, more preferably between 5 and 14, even more preferably between 5 and 12, even more preferably still 5 or 6 carbon atoms, with one or more carbon-carbon double bonds, preferably with 1, 2 or 3 carbon-carbon double bonds, conjugated or unconjugated, which is bound to the rest of the molecule through a single bond, including, for example and not restricted to, the cyclopent-1-en-1-yl group and similar groups.

The term “cycloalkynyl” relates to a mono- or polycyclic aliphatic group which has between 5 and 24, preferably between 5 and 16, more preferably between 5 and 14, even more preferably between 5 and 12, even more preferably still 5 or 6 carbon atoms, with one or more carbon-carbon triple bonds, preferably with 1, 2 or 3 carbon-carbon triple bonds, conjugated or unconjugated, which is bound to the rest of the molecule through a single bond, including, for example and not restricted to, the cyclohex-1-yn-1-yl group and similar groups.

The term “aryl group” relates to an aromatic group which has between 6 and 30, preferably between 6 and 18, more preferably between 6 and 10, even more preferably 6 or 10 carbon atoms, which comprise 1, 2, 3 or 4 aromatic rings, bound by a carbon-carbon bond or fused, including, for example and not restricted to, phenyl, naphthyl, diphenyl, indenyl, phenanthryl or anthranyl among others; or an aralkyl group.

The term “aralkyl group” relates to an alkyl group substituted with an aromatic group, with between 7 and 24 carbon atoms and including, for example and not restricted to, —(CH₂)₁₋₆-phenyl, —(CH₂)₁₋₆-(1-naphthyl), —(CH₂)₁₋₆-(2-naphthyl), —(CH₂)₁₋₆—CH(phenyl)₂ and similar.

The term “heterocyclic group” relates to a 3-10 member hydrocarbon ring, in which one or more of the ring atoms, preferably 1, 2 or 3 of the ring atoms, is a different element to carbon, such as nitrogen, oxygen or sulfur and may be saturated or unsaturated. For the purposes of this invention, the heterocycle can be a cyclic, monocyclic, bicyclic or tricyclic system which may include fused ring systems; and the nitrogen, carbon or sulfur atoms can be optionally oxidized in the heterocyclyl radical; the nitrogen atom can optionally be quaternized; and the heterocyclyl radical may be partially or completely saturated or may be aromatic. With increasing preference, the term heterocyclic relates to a 5 or 6 member ring.

The term “heteroarylalkyl group” relates to an alkyl group substituted with a substituted or unsubstituted aromatic heterocyclyl group, the alkyl group having from 1 to 6 carbon atoms and the aromatic heterocyclyl group between 2 and 24 carbon atoms and from 1 to 3 atoms other than carbon and including, for example and not restricted to, —(CH₂)₁₋₆-imidazolyl, —(CH₂)₁₋₆-triazolyl, —(CH₂)₁₋₆-thienyl, —(CH₂)₁₋₆-furyl, —(CH₂)₁₋₆₋pyrrolidinyl and similar

As used in this technical area, there may be a degree of substitution on the groups defined above. Thus, there can be substitution in any of the groups of this invention. The references in this document to groups substituted in the groups of this invention indicate that the radical specified can be substituted in one or more available positions by one or more substituents, preferably in 1, 2 or 3 positions, more preferably in 1 or 2 positions, even more preferably in 1 position. These substituents include, for example and not restricted to, alkyl C₁-C₄; hydroxyl; alkoxyl C₁-C₄; amino; aminoalkyl C₁-C₄; carbonyloxyl C₁-C₄; oxycarbonyl C₁-C₄; halogen such as fluorine, chlorine, bromine and iodine; cyano; nitro; azido; alkylsulfonyl C₁-C₄; thiol; alkylthio C₁-C₄, aryloxyl such as phenoxyl; —NR_{b}(C═NR_{b})NR_{b}R_{c}; where R_{b} and R_{c} are selected independently from the group consisting of H, alkyl C₁-C₄, alkenyl C₂-C₄, alkynyl C₂-C₄, cycloalkyl C₃-C₁₀, aryl C₆-C₁₈, aralkyl C₇-C₁₇, 3-10-membered-heterocyclyl or protective group of the amino group.

### Compounds of the Invention

The compounds of the invention are defined by the general formula (I)

R₁-AA₁-AA₂-AA₃-R₂ (I)
their stereoisomers, mixtures thereof and/or their cosmetically or pharmaceutically acceptable salts, characterized in that:
AA₁ and AA₂ are independently selected from amongst themselves from the group consisting of -Tyr- and -Phe-;
AA₃ is selected from the group consisting of -Nle- and -Met-;
R₁ is selected from the group consisting of H, substituted or unsubstituted non-cyclic aliphatic group, substituted or unsubstituted alicyclyl, substituted or unsubstituted heterocyclyl, substituted or unsubstituted heteroarylalkyl, substituted or unsubstituted aryl, substituted or unsubstituted aralkyl and R₅—CO—; and
R₂ is selected from the group consisting of —NR₃R₄, —OR₃ and —SR₃;
where R₃ and R₄ are independently selected from the group consisting of H, substituted or unsubstituted non-cyclic aliphatic group, substituted or unsubstituted alicyclyl, substituted or unsubstituted heterocyclyl, substituted or unsubstituted heteroarylalkyl, substituted or unsubstituted aryl, and substituted or unsubstituted aralkyl;
and where R₅ is selected from the group consisting of H, substituted or unsubstituted non-cyclic aliphatic group, substituted or unsubstituted alicyclyl, substituted or unsubstituted aryl, substituted or unsubstituted aralkyl, substituted or unsubstituted heterocyclyl and substituted or unsubstituted heteroarylalkyl;
The R₁ and R₂ groups are bound to the amino-terminal (N-terminal) and carboxy-terminal (C-terminal) ends of the peptide sequences respectively.

According to a preferred embodiment of this invention, R₁ is selected from the group consisting of H or R₅—CO—, wherein R₅ is selected from the group consisting of substituted or unsubstituted alkyl radical C₁-C₂₄, substituted or unsubstituted alkenyl C₂-C₂₄, substituted or unsubstituted alkynyl C₂-C₂₄, substituted or unsubstituted cycloalkyl C₅-C₂₄, substituted or unsubstituted cycloalkenyl C₅-C₂₄, substituted or unsubstituted cycloalkynyl C₅-C₂₄, substituted or unsubstituted aryl C₆-C₃₀, substituted or unsubstituted aralkyl C₇-C₂₄, substituted or unsubstituted heterocyclyl with 3-10 ring members, and substituted or unsubstituted heteroarylalkyl of 2 to 24 carbon atoms and 1 to 3 atoms other than carbon and an alkyl chain of 1 to 6 carbon atoms. More preferably, R₁ is selected from H, acetyl, tert-butanoyl, hexanoyl, 2-methylhexanoyl, cyclohexancarboxyl, octanoyl, decanoyl, lauroyl, myristoyl, palmitoyl, stearoyl, oleoyl and linoleoyl. Even more preferably, R₁ is H, acetyl, lauroyl, myristoyl or palmitoyl.

In an even more preferred embodiment, R₁ is acetyl or palmitoyl.

According to another preferred embodiment, R₂ is —NR₃R₄, —OR₃ or —SR₃, wherein R₃ and R₄ are independently selected from the group consisting of H, substituted or unsubstituted alkyl C₁-C₂₄, substituted or unsubstituted alkenyl C₂-C₂₄, substituted or unsubstituted alkynyl C₂-C₂₄, substituted or unsubstituted cycloalkyl C₃-C₂₄, substituted or unsubstituted cycloalkenyl C₅-C₂₄, substituted or unsubstituted cycloalkynyl C₅-C₂₄, substituted or unsubstituted aryl C₆-C₃₀, substituted or unsubstituted aralkyl C₇-C₂₄, substituted or unsubstituted heterocyclyl with 3-10 ring members and substituted or unsubstituted heteroarylalkyl of 2 to 24 carbon atoms and 1 to 3 atoms other than carbon and an alkyl chain of 1 to 6 carbon atoms. Optionally, R₃ and R₄ can be bound through a saturated or unsaturated carbon-carbon bond, forming a cycle with the nitrogen atom. More preferably R₂ is —NR₃R₄, or —OR₃, wherein R₃ and R₄ are independently selected from the group consisting of H, substituted or unsubstituted alkyl C₁-C₂₄, substituted or unsubstituted alkenyl C₂-C₂₄, substituted or unsubstituted alkynyl C₂-C₂₄, substituted or unsubstituted cycloalkyl C₃-C₁₀, substituted or unsubstituted aryl C₆-C₁₅ and substituted or unsubstituted heterocyclyl with 3-10 ring members, substituted or unsubstituted heteroarylalkyl with 3 to 10 ring members and an alkyl chain of 1 to 6 carbon atoms. More preferably R₃ and R₄ are selected from the group consisting of H, methyl, ethyl, hexyl, dodecyl or hexadecyl. Even more preferably R₃ is H and R₄ is selected from the group consisting of H, methyl, ethyl, hexyl, dodecyl or hexadecyl. According to an even more preferable embodiment, R₂ is selected from —OH and —NH₂.

According to another embodiment of this invention R₁ is selected from the group consisting of H, acetyl, lauroyl, myristoyl or palmitoyl, AA₁ is -L-Tyr-, AA₂ is -L-Tyr-, AA₃ is -L-Met-, and R₂ is —NR₃R₄ or —OR₃ wherein R₃ and R₄ are independently selected from H, methyl, ethyl, hexyl, dodecyl and hexadecyl, preferably R₂ is —OH or —NH₂. More preferably, R₁ is acetyl or palmitoyl and R₂ is —NH₂.

According to another embodiment of this invention R₁ is selected from the group consisting of H, acetyl, lauroyl, myristoyl or palmitoyl, AA₁ is -L-Tyr-, AA₂ is -L-Phe-, AA₃ is -L-Met-, and R₂ is —NR₃R₄ or —OR₃ wherein R₃ and R₄ are independently selected from H, methyl, ethyl, hexyl, dodecyl and hexadecyl, preferably R₂ is —OH or —NH₂. More preferably, R₁ is acetyl or palmitoyl and R₂ is —NH₂.

According to another embodiment of this invention R₁ is selected from the group consisting of H, acetyl, lauroyl, myristoyl or palmitoyl, AA₁ is -L-Tyr-, AA₂ is -L-Tyr-, AA₃ is -L-Nle-, and R₂ is —NR₃R₄ or —OR₃ wherein R₃ and R₄ are independently selected from H, methyl, ethyl, hexyl, dodecyl and hexadecyl, preferably R₂ is —OH or —NH₂. More preferably, R₁ is acetyl or palmitoyl and R₂ is —NH₂.

According to another embodiment of this invention R₁ is selected from the group consisting of H, acetyl, lauroyl, myristoyl and palmitoyl, preferably R₁ is selected from the group consisting of H, acetyl and palmitoyl and R₂ is selected from the group consisting of —OH and —NH₂.

Preferably, the compounds of formula (I) are selected from the group consisting of:
Palm-Tyr-Tyr-Met-NH₂,
Palm-Tyr-Tyr-Met-OH,
Ac-Tyr-Tyr-Met-NH₂,
Ac-Tyr-Tyr-Met-OH,
Ac-Tyr-Tyr-Met-NH—(CH₂)₁₅—CH₃,
Palm-Tyr-Phe-Met-NH₂,
Palm-Tyr-Phe-Met-OH,
Ac-Tyr-Phe-Met-NH₂,
Ac-Tyr-Phe-Met-OH,
Ac-Tyr-Phe-Met-NH—(CH₂)₁₅—CH₃,
Palm-Phe-Tyr-Met-NH₂,
Palm-Phe-Tyr-Met-OH,
Ac-Phe-Tyr-Met-NH₂,
Ac-Phe-Tyr-Met-OH,
Ac-Phe-Tyr-Met-NH—(CH₂)₁₅—CH₃,
Palm-Tyr-Tyr-Nle-NH₂,
Palm-Tyr-Tyr-Nle-OH,
Ac-Tyr-Tyr-Nle-NH₂,
Ac-Tyr-Tyr-Nle-OH, and
Ac-Tyr-Tyr-Nle-NH—(CH₂)₁₅—CH₃;
   their stereoisomers, mixtures thereof and/or their cosmetically or pharmaceutically acceptable salts.

The peptides of this invention can exist as stereoisomers or mixtures of stereoisomers; for example, the amino acids which form them can have an L-, D-configuration or be racemic independently of one another. Therefore, it is possible to obtain isomeric mixtures as well as racemic mixtures or diastereomeric mixtures, or pure diastereomers or enantiomers, depending on the number of asymmetric carbons and which isomers or isomeric mixtures are present. The preferred structures of the peptides of the invention are pure isomers, i.e., enantiomers or diastereomers.

For example, when it is indicated that AA₁ can be -Tyr, it is understood that AA₁ is selected from -L-Tyr-, -D-Tyr- or mixtures of both, racemic or non-racemic. Likewise, when it is said that AA₂ can be -Met-, it is understood that it can be -L-Met-, -D-Met- or mixtures of both, racemic or non-racemic. The preparation processes described in this document allow the person skilled in the art to obtain each of the stereoisomers of the peptide of the invention by choosing the amino acid with the appropriate configuration.

In the context of this invention there are also cosmetically or pharmaceutically acceptable salts of the peptides provided by this invention. The term “cosmetically or pharmaceutically acceptable salts” means a salt admitted for its use in animals and, more particularly, human beings, and includes the salts used to form base addition salts, whether inorganic, such as and not restricted to, lithium, sodium, potassium, calcium, magnesium, manganese, copper, zinc or aluminum among others; or organic such as and not restricted to, ethylamine, diethylamine, ethylenediamine, ethanolamine, diethanolamine, arginine, lysine, histidine or piperazine among others; or acid addition salts, whether organic, such as and not restricted to, acetate, citrate, lactate, malonate, maleate, tartrate, fumarate, benzoate, aspartate, glutamate, succinate, oleate, trifluoroacetate, oxalate, pamoate or gluconate among others; or inorganic, such as and not restricted to chloride, sulfate, borate or carbonate among others. The nature of the salt is not critical, provided that it is cosmetically and pharmaceutically acceptable. Cosmetically and pharmaceutically acceptable salts of the peptides of the invention can be obtained by conventional methods, well known in the prior art [Berge S. M., Bighley L. D. and Monkhouse D. C. (1977) “Pharmaceutical Salts” J. Pharm. Sci. 66:1-19].

Another aspect of this invention relates to a peptide of general formula (I), its stereoisomers, mixtures thereof, and/or its cosmetically or pharmaceutically acceptable salts, as described in this invention, for the treatment and/or care of the skin and/or hair.

In another particular aspect, this invention relates to a peptide of general formula (I), its stereoisomers, mixtures thereof, and/or its cosmetically or pharmaceutically acceptable salts, as described in this invention, for the treatment, prevention and/or care of those conditions, disorders and/or diseases of the skin and/or hair which require cAMP synthesis stimulation.

In another particular aspect, this invention relates to a peptide of general formula (I), its stereoisomers, mixtures thereof, and/or its cosmetically or pharmaceutically acceptable salts, as described in this invention, for the treatment of the skin and/or hair which stimulates melanin synthesis in the skin and/or hair.

In another particular aspect, this invention relates to a peptide of general formula (I), its stereoisomers, mixtures thereof, and/or its cosmetically or pharmaceutically acceptable salts, as described in this invention, for the treatment of the skin and/or hair, which accelerates, intensifies and/or prolongs the skin's tan.

In another particular aspect, this invention relates to a peptide of general formula (I), its stereoisomers, mixtures thereof, and/or its cosmetically or pharmaceutically acceptable salts, as described in this invention, for the treatment of the skin and/or hair, which reduces pigmentation irregularities, preferably irregularities caused by vitiligo.

In another particular aspect, this invention relates to a peptide of general formula (I), its stereoisomers, mixtures thereof, and/or its cosmetically or pharmaceutically acceptable salts, as described in this invention, for the treatment of the skin and/or hair, which reduces, delays or prevents damage induced by UV radiation.

In another particular aspect, this invention relates to a peptide of general formula (I), its stereoisomers, mixtures thereof, and/or its cosmetically or pharmaceutically acceptable salts, as described in this invention, for the treatment of the skin and/or hair, which reduces, delays or prevents the signs of aging and/or photoaging.

In another particular aspect, this invention relates to a peptide of general formula (I), its stereoisomers, mixtures thereof, and/or its cosmetically or pharmaceutically acceptable salts, as described in this invention, for the treatment of the skin, which stimulates lipolysis.

In another particular aspect, this invention relates to a peptide of general formula (I), its stereoisomers, mixtures thereof, and/or its cosmetically or pharmaceutically acceptable salts, as described in this invention, for the treatment of the skin, which reduces, delays and/or prevents cellulite.

In another particular aspect, the treatment and/or care of this invention is performed by topical or transdermal application; preferably, the topical or transdermal application is performed via iontophoresis, sonophoresis, electroporation, mechanical pressure, osmotic pressure gradient, occlusive cure, microinjections, needle-free injections by means of pressure, by means of microelectric patches or any combination thereof.

In another particular aspect, the treatment and/or care is performed by oral administration.

### Processes of Preparation

The synthesis of the peptides of the invention, their stereoisomers or their cosmetically or pharmaceutically acceptable salts can be performed according to conventional methods known in the prior art, such as methods of solid phase peptide synthesis [Stewart J. M. and Young J. D. (1984) “Solid Phase Peptide Synthesis, 2nd edition” Pierce Chemical Company, Rockford, Ill.; Bodanzsky M. and Bodanzsky A. (1984) “The practice of Peptide Synthesis” Springer Verlag, New Cork; Lloyd-Williams P., Albericio F. and Giralt E. (1997) “Chemical Approaches to the Synthesis of Peptides and Proteins” CRC, Boca Raton, Fla., USA], methods of synthesis in solution, a combination of the methods for solid phase synthesis and solution synthesis or methods of enzymatic synthesis [Kullmann W. (1980) “Proteases as catalysts for enzymic syntheses of opioid peptides” J. Biol. Chem. 255:8234-8238]. The peptides can also be obtained by fermentation of a bacterial strain, genetically engineered or not, in order to produce the desired sequences, by controlled hydrolysis of proteins of animal or vegetable origin, preferably vegetable origin, to release peptide fragments containing at least the desired sequence.

For example, a method of obtaining the peptides of the invention of formula (I) comprises the steps of:
coupling an amino acid with the N-terminal end protected and the C-terminal end free, onto an amino acid with the N-terminal end free and the C-terminal end protected or bound to a solid support;
removing the protective group of the N-terminal end;
repeating of the sequence of coupling and removal of the protective group of the N-terminal end until the desired peptidic sequence is obtained;
removal of the protective group of the C-terminal end or cleavage from the solid support.

Preferably, the C-terminal end is bound to a solid support and the process is conducted on solid phase and, therefore, includes the coupling of an amino acid with the N-terminal end protected and the C-terminal end free onto an amino acid with the N-terminal end free and the C-terminal end bound to a polymer support; removal of the protective group of the N-terminal end; and repetition of this sequence as many times as is necessary to obtain a peptide of the desired length, and finally followed by cleaving the synthesized peptide from the original polymer support.

The functional groups of the side chains of the amino acids are adequately protected with temporary or permanent protective groups throughout synthesis, and can be deprotected simultaneously or orthogonally to the process of cleaving the peptide from the polymer support.

Alternatively, solid phase synthesis can be carried out by a convergent strategy coupling a peptide onto the polymer support or onto an amino acid previously bound to the polymer support. Convergent synthesis strategies are widely known to the person skilled in the art and are described in Lloyd-Williams P., Albericio F. and Giralt E. in “Convergent solid-phase peptide synthesis” (1993) Tetrahedron 49:11065-11133.

The process can comprise the additional stages of deprotection of the N-terminal and C-terminal ends and/or cleavage of the peptide from the polymer support in a different order, using standard processes and conditions known in the prior art, after which the functional groups of these ends can be modified. The optional modification of the N-terminal and C-terminal ends can be carried out with the peptide of formula (I) bound to the polymeric support or once the peptide has been cleaved from the polymeric support.

Alternatively, R₁ may be introduced by the reaction of the N-terminal end of the peptide of the invention with a compound R₁—X, wherein R₁ has the meaning described above and X is a leaving group such as and not restricted to, the tosyl group, the mesyl group and halogen groups among others; through a nucleophilic substitution reaction, in the presence of an adequate base and solvent, wherein the fragments that have the functional groups not involved in the N—C bond formation are suitably protected with temporary or permanent protective groups.

Optionally and/or additionally, the R₂ radicals can be introduced by the reaction of a compound HR₂ wherein R₂ is —OR₃, —NR₃R₄ or —SR₃, with a complementary fragment which corresponds to the peptide of formula (I) in which R₂ is —OH in the presence of an adequate solvent and a base such as, N,N-diisopropylethylamine (DIEA) or triethylamine or an additive such as 1-hydroxybenzotriazole (HOBt) or 1-hydroxyazabenzotriazole (HOAt) and a dehydrating agent, such as a carbodiimide, an uronium salt, a phosphonium salt or amidinium salt, among others, or by prior formation of an acyl halide with, for example, thionyl chloride, and thereby obtaining a peptide according to the general formula (I) invention, wherein the fragments that have the functional groups not involved in the N—C bond formation are suitably protected with temporary or permanent protective groups, or alternatively other R₂ radicals may be introduced by simultaneous incorporation to the peptide cleavage process from the polymeric support.

A person skilled in the art would easily understand that the deprotection/cleavage steps of the C-terminal and N-terminal ends and their subsequent derivatization can be performed in a different order, according to the processes known in the prior art [Smith M. B. and March J. (1999) “March's Advanced Organic Chemistry Reactions, Mechanisms and Structure”, 5th Edition, John Wiley & Sons, 2001].

The term “protective group” relates to a group which blocks an organic functional group and can be removed in controlled conditions. The protective groups, their relative reactivities and the conditions in which they remain inert are known to the person skilled in the art.

Examples of protective groups representative for the amino group are amides, such as amide acetate, amide benzoate, amide pivalate; carbamates such as benzyloxycarbonyl (Cbz or Z), 2-chlorobenzyl (CIZ), para-nitrobenzyloxycarbonyl (pNZ), tert-butyloxycarbonyl

(Boc), 2,2,2-trichloroethyloxycarbonyl (Troc), 2-(trimethylsilyl)ethyloxycarbonyl (Teoc), 9-fluorenylmethyloxycarbonyl (Fmoc) or allyloxycarbonyl (Alloc), Trityl (Trt), methoxytrityl (Mtt), 2,4-dinitrophenyl (Dnp), N-[1-(4,4-dimethyl-2,6-dioxocyclohex-1-ylidene)ethyl (Dde), dimethyl-2,6-dioxo-cyclohexylidene)-3-methylbutyl (ivDde), 1-(1-adamantyl)-1-methylethoxycarbonyl (Adpoc), among others, preferably Boc or Fmoc.

Examples of protective groups representative for the carboxyl group are esters, such as the tert-butyl ester (tBu), allyl ester (All), triphenylmethyl ester (trityl ester, Trt), cyclohexyl ester (cHex), benzyl ester (Bzl), ortho-nitrobenzyl ester, para-nitrobenzyl ester, para-methoxybenzyl ester, trimethylsilylethyl ester, 2-phenylisopropyl ester, fluorenylmethyl ester (Fm), 4-(N-[1-(4,4-dimethyl-2,6-dioxocyclohexylidene)-3-methylbutyl]amino) benzyl ester (Dmab), among others; preferred protective groups of the invention are the All, tBu, cHex, Bzl and Trt esters.

The side chains of the trifunctional amino acids can be protected during the synthetic process with temporary or permanent protective groups orthogonal to the protective groups of the N-terminal and C-terminal ends.

The hydroxyl group of the tyrosine side chain can be protected with the 2-bromobenzyloxycarbonyl group (2-BrZ), tert-butyl (tBu), allyl (All), benzyl (Bzl) or 2,6-dichlorobenzyl (2,6-diCIZ) among others. The methionine side chain can be protected by as a sulfoxide or can be used unprotected.

In a preferred embodiment, the protective group strategy used is the strategy wherein the amino groups are protected by Boc, the carboxyl groups are protected by Bzl, cHex or All, the tyrosine side chain is protected with 2-BrZ or Bzl and methionine side chain is used unprotected.

In another preferred embodiment, the protective group strategy used is the strategy wherein the amino groups are protected by Fmoc, the carboxyl groups are protected by tBu, All or Trt, the tyrosine side chain is protected with tBu and the methionine side chain is used unprotected.

Examples of these and other additional protective groups, their introduction and removal, can be found in the literature [Greene T. W. and Wuts P. G. M., (1999) “Protective groups in organic synthesis” John Wiley & Sons, New York; Atherton B. and Sheppard R. C. (1989) “Solid Phase Peptide Synthesis: A practical approach” IRL Oxford University Press]. The term “protective groups” also includes the polymeric supports used in solid phase synthesis.

When the synthesis takes place totally or partially on solid phase, the possible solid supports used in the method of the present invention involve polystyrene supports, polyethylene glycol grafted to polystyrene and similar, such as and not restricted to, p-methylbenzhydrylamine (MBNA) resins [Matsueda G. R. and Stewart J. M. 1981) “A p-methylbenzhydrylamine resin for improved solid-phase synthesis of peptide amides” Peptides 2:45-50], 2-chlorotrityl resins [Barlos K., Gatos D., Kallitsis J., Papaphotiu G., Sotiriu P., Wenqing Y. and Schäfer W. (1989) “Darstellung geschützter Peptid-Fragmente unter Einsatz substituierter Triphenylmethyl-Harze” Tetrahedron Lett. 30:3943-3946; Barlos K., Gatos D., Kapolos S., Papaphotiu G., Schäfer W. and Wenqing Y. (1989) “Veresterung von partiell geschützten Peptid-Fragmenten mit Harzen. Einsatz von 2-Chlorotritylchlorid zur Synthese von Leu1-Gastrin I” Tetrahedron Lett. 30:3947-3951], TentaGel® resins (Rapp Polymere GmbH), ChemMatrix® resins (Matrix Innovation, Inc) and similar, which may or not include a labile linker, such as 5-(4-aminomethyl-3,5-dimethoxyphenoxy)valeric acid (PAL) [Albericio F., Kneib-Cordonier N., Biancalana S., Gera L., Masada R. I., Hudson D. and Barany G. (1990) “Preparation and application of the 5-(4-(9-fluorenylmethyloxycarbonyl)aminomethyl-3,5-dimethoxy-phenoxy)valeric acid (PAL) handle for the solid-phase synthesis of C-terminal peptide amides under mild conditions” J. Org. Chem. 55:3730-3743],2-28:3787-3790], Wang [Wang S. S. (1973) “p-Alkoxybenzyl Alcohol Resin and p-Alkoxybenzyloxycarbonylhydrazide Resin for Solid Phase Synthesis of Protected Peptide Fragments” J. Am. Chem. Soc. 95:1328-1333] and similar, allowing the simultaneous deprotection and cleavage of the peptide from the polymeric support.

### Cosmetic or Pharmaceutical Compositions

The peptides of the invention can be administered to stimulate melanin synthesis by any means which produces the peptides' contact with their site of action in the body of a mammal, preferably human, and in the form of a composition that contains them.

To this regard, another aspect of the invention is a cosmetic or pharmaceutical composition which comprises at least a peptide of general formula (I), its stereoisomers, mixtures thereof, and/or its cosmetically or pharmaceutically acceptable salts together with at least one cosmetically or pharmaceutically acceptable adjuvant. These compositions can be prepared by conventional means known to persons skilled in the art (“Harry's Cosmeticology”, Eight edition (2000) Rieger M. M., ed., New York Chemical Pub., NY, US; “Remington: The Science and Practice of Pharmacy”, Twentieth edition (2003) Genaro A. R., ed., Lippincott Williams & Wilkins, Philadelphia, US].

The peptides of this invention have variable solubility in water, according to the nature of their sequence or any possible modifications in the N-terminal and/or C-terminal ends. Therefore, the peptides of this invention can be incorporated into the compositions by aqueous solution, and those which are not soluble in water can be solubilized in cosmetically or pharmaceutically acceptable conventional solvents such as and not restricted to, ethanol, propanol, isopropanol, propylene glycol, glycerine, butylene glycol or polyethylene glycol or any combination thereof.

The cosmetically or pharmaceutically effective amount of the peptides of the invention which should be administered, as well as their dosage, will depend on numerous factors, including age, state of the patient, the nature or severity of the condition, disorder or disease to be treated and/or care for, the route and frequency of administration and of the particular nature of the peptides to be used.

“Cosmetically and pharmaceutically effective amount” is understood to mean a non-toxic but sufficient amount of the peptide or peptides of the invention to provide the desired effect. The peptides of the invention are used in the cosmetic or pharmaceutical composition of this invention in cosmetically or pharmaceutically effective concentrations to achieve the desired effect; in a preferred form versus the total weight of the composition, between 0.00000001% (in weight) and 20% (in weight); preferably between 0.000001% (in weight) and 20% (in weight), more preferably between 0.0001% (in weight) and 10% (in weight) and even more preferably between 0.0001% (in weight) and 5% (in weight).

The peptides of the invention can also be incorporated into cosmetic or pharmaceutical delivery systems and/or sustained release systems.

The term “delivery systems” relates to a diluent, adjuvant, excipient or carrier with which the peptide of the invention is administered. These cosmetic or pharmaceutical carriers can be liquids, such as water, oils or surfactants, including those of petroleum, animal, vegetable or synthetic origin, such as and not restricted to, peanut oil, soybean oil, mineral oil, sesame oil, castor oil, polysorbates, sorbitan esters, ether sulfates, sulfates, betaines, glycosides, maltosides, fatty alcohols, nonoxynols, poloxamers, polyoxyethylenes, polyethylene glycols, dextrose, glycerol, digitonin and similar. In “Remington's Pharmaceutical Sciences” by E. W. Martin diluents, adjuvants or excipients are described as appropriate carriers.

The term “sustained release” is used in a conventional sense relating to a delivery system of a compound which provides the gradual release of this compound during a period of time and preferably, although not necessarily, with relatively constant compound release levels over a period of time.

Examples of delivery or sustained release systems are liposomes, mixed liposomes, oleosomes, niosomes, miniparticles, milliparticles, microparticles, nanoparticles and solid lipid nanoparticles, nanostructured lipid carriers, sponges, cyclodextrins, vesicles, micelles, mixed micelles of surfactants, surfactant-phospholipid mixed micelles, millispheres, microspheres and nanospheres, lipospheres, millicapsules, microcapsules and nanocapsules, as well as microemulsions and nanoemulsions, which can be added to achieve a greater penetration of the active principle and/or improve its pharmacokinetic and pharmacodynamic properties. Preferred delivery or sustained release systems are liposomes, surfactant-phospholipid mixed micelles and microemulsions, more preferably water-in-oil microemulsions with an internal structure of reverse micelle.

The sustained release systems can be prepared by methods known in the prior art, and the compositions which contain them can be administered, for example, by topical administration, including adhesive patches, non-adhesive patches and microelectric patches, or by systemic administration, for example and not restricted to, orally or parenterally, including nasal, rectal or subcutaneous implantation or injection, or direct implantation or injection into a specific body part, and preferably should release a relatively constant quantity of the peptides of the invention. The amount of peptide contained in the sustained release system will depend, for example, on where the composition is to be administered, the kinetics and duration of the release of the peptide of the invention, as well as the nature of the condition, disorder and/or disease to be treated and/or cared for.

The peptides of this invention can also be adsorbed on solid organic polymers or solid mineral supports such as and not restricted to, talc, bentonite, silica, starch or maltodextrin among others.

The compositions which contain the peptides of the invention can also be incorporated into fabrics, non-woven fabrics and medical devices which are in direct contact with the skin and/or hair, thus releasing the peptides of the invention whether by biodegradation of the binding system to the fabric, non-woven fabric or medical device, or by the friction between them and the body, due to body moisture, the skin's pH or body temperature. Furthermore, the fabrics and non-woven fabrics can be used for making garments that are in direct contact with the body. Preferably, the fabrics, non-woven fabrics and medical devices containing peptides of the invention are used for the treatment and/or care of those conditions, disorders and/or diseases of the skin and/or hair which require cAMP synthesis stimulation.

Examples of fabrics, non-woven fabrics, garments, medical devices and means for immobilizing the peptides to them, among which are the delivery systems and/or the sustained release systems described above, can be found in literature and are known in the prior art [Schaab C. K. (1986) “Impregnating Fabrics With Microcapsules”, HAPPI May 1986; Nelson G. (2002) “Application of microencapsulation in textiles” Int. J. Pharm. 242:55-62; “Biofunctional Textiles and the Skin” (2006) Curr. Probl. Dermatol. v.33, Hipler U. C. and Elsner P., eds. S. Karger AG, Basel, Switzerland; Malcom R. K.; McCullagh S. D., Woolfson A. D., Gorman S. P., Jones D. S, and Cuddy J. (2004) “Controlled release of a model antibacterial drug from a novel self-lubricating silicone biomaterial” J. Cont. Release 97:313-320]. The preferred fabrics, non-woven fabrics, garments and medical devices are bandages, gauzes, t-shirts, socks, tights, underwear, girdles, gloves, diapers, sanitary napkins, dressings, bedspreads, wipes, adhesive patches, non-adhesive patches, microelectric patches and/or face masks.

The cosmetic or pharmaceutical compositions which contain the peptides of this invention, their stereoisomers, mixtures thereof and/or their cosmetically or pharmaceutically acceptable salts, can be used in different types of compositions of topical or transdermal application, optionally including cosmetically or pharmaceutically acceptable excipients necessary for formulating the desired administration form Faulí i Trillo C. (1993) in “Tratado de Farmacia Galénica”, Luzán 5, S. A. Ediciones, Madrid].

The compositions of topical or transdermal application can be produced in any solid, liquid or semisolid formulation, such as and not restricted to, creams, multiple emulsions such as and not restricted to, oil and/or silicone in water emulsions, water-in-oil and/or silicone emulsions, water/oil/water or water/silicone/water type emulsions, and oil/water/oil or silicone/water/silicone type emulsions, anhydrous compositions, aqueous dispersions, oils, milks, balsams, foams, lotions, gels, cream gels, hydroalcoholic solutions, hydroglycolic solutions, hydrogels, liniments, sera, soaps, shampoos, conditioners, serums, polysaccharide films, ointments, mousses, pomades, powders, bars, pencils and sprays or aerosols (sprays), including leave-on and rinse-off formulations. These topical or transdermal application formulations can be incorporated using techniques known by the person skilled in the art into different types of solid accessories such as and not restricted to, wipes, adhesive patches, non-adhesive patches, microelectric patches or face masks, or they can be incorporated into different make-up products such as make-up foundation, such as fluid foundations and compact foundations, make-up removal lotions, make-up removal milks, under-eye concealers, eye shadows, lipsticks, lip protectors, lip gloss and powders among others.

The cosmetic and pharmaceutical compositions of the invention may include agents which increase the percutaneous absorption of the peptides of this invention, such as and not restricted to, dimethylsulfoxide, dimethylacetamide, dimethylformamide, surfactants, azone (1-dodecylazacycloheptane-2-one), alcohol, urea, ethoxydiglycol, acetone, propylene glycol or polyethylene glycol, among others. Furthermore, the cosmetic or pharmaceutical compositions of this invention can be applied to local areas to be treated by means of iontophoresis, sonophoresis, electroporation, microelectric patches, mechanical pressure, osmotic pressure gradient, occlusive cure, microinjections or needle-free injections by means of pressure, such as injections by oxygen pressure, or any combination thereof, to achieve a greater penetration of the peptide of the invention. The application area will be determined by the nature of the condition, disorder and/or disease to be treated and/or cared for.

Furthermore, the cosmetic compositions containing the peptides of this invention, their stereoisomers and/or their cosmetically or pharmaceutically acceptable salts can be used in different types of formulations for oral administration, preferably in the form of oral cosmetics, such as and not restricted to, capsules, including gelatin capsules, tablets, including sugar coated tablets, powders, granules, chewing gum, solutions, suspensions, emulsions, syrups, polysaccharide films, jellies or gelatins, and any other form known by the person skilled in the art. In particular, the peptides of the invention can be incorporated into any form of functional food or fortified food, such as and not restricted to, dietary bars or compact or non-compact powders. These powders can be dissolved in water, juices, soda, dairy products, soya derivatives or can be incorporated into dietary bars. The peptides of this invention can be formulated with common excipients and adjuvants for oral compositions or food supplements, such as and not restricted to, fat components, aqueous components, humectants, preservatives, texturizing agents, flavors, aromas, antioxidants and colorants common in the food industry.

Cosmetic or pharmaceutical compositions containing the peptides of the invention, their stereoisomers, mixtures thereof and/or their cosmetically or pharmaceutically acceptable salts can also be administered by topical or transdermal route, as well as by any other appropriate route, as for example oral or parenteral route, for which they will include the pharmaceutically acceptable excipients necessary for the formulation of the desired administration form. In the context of this invention, the term “parenteral” includes nasal, auricular, ophthalmic, vaginal and rectal route, subcutaneous, intradermal, intravascular injections, such as intravenous, intramuscular, intravitreous, intraspinal, intracranial, intraarticular, intrathecal and intraperitoneal injections and any another similar injection or infusion technique. A review of the different pharmaceutical forms of administration of the active ingredients and excipients necessary for obtaining them can be found, for example, in the “Tratado de Farmacia Galénica”, C. Faulí i Trill®, 1993, Luzán 5, S. A. Ediciones, Madrid.

Among the cosmetically or pharmaceutically acceptable adjuvants contained in the cosmetic or pharmaceutical compositions described in this invention include additional ingredients commonly used in compositions for the treatment and/or care of the skin and/or hair such as and not restricted to, other cAMP synthesis stimulating agents, matrix metalloproteinase inhibiting agents, melanin synthesis stimulating or inhibiting agents, whitening or depigmenting agents, propigmenting agents, self-tanning agents, antiaging agents, NO-synthase inhibiting agents, 5α-reductase inhibiting agents, lysyl- and/or prolyl hydroxylase inhibiting agents, antioxidants, free radical scavengers and/or agents against atmospheric pollution, reactive carbonyl species scavengers, anti-glycation agents, antihistamine agents, antiemetic agents, antiviral agents, antiparasitic agents, emulsifiers, emollients, organic solvents, liquid propellants, skin and/or hair conditioners such as humectants, substances that retain moisture, alpha hydroxyacids, beta hydroxyacids, moisturizers, epidermal hydrolytic enzymes, vitamins, pigments or colorants, dyes, gelling polymers, thickeners, surfactants, softening agents, anti-wrinkle agents, agents able to reduce or treat bags under the eyes, exfoliating agents, antimicrobial agents, antifungal agents, fungistatic agents, bactericidal agents, bacteriostatic agents, agents stimulating the synthesis of dermal or epidermal macromolecules and/or capable of inhibiting or preventing their degradation, such as for example collagen synthesis-stimulating agents, elastin synthesis-stimulating agents, decorin synthesis-stimulating agents, laminin synthesis-stimulating agents, defensin synthesis-stimulating agents, chaperone synthesis-stimulating agents, aquaporin synthesis-stimulation agents, hyaluronic acid synthesis-stimulating agents, fibronectin synthesis-stimulating agents, sirtuin synthesis-stimulating agents, agents stimulating the synthesis of lipids and components of the stratum corneum (ceramides, fatty acids, etc.), agents that inhibit collagen degradation, other agents that inhibit elastin degradation, agents that inhibit serine proteases such cathepsin G, agents stimulating fibroblast proliferation, agents stimulating keratinocyte proliferation, agents stimulating adipocyte proliferation, agents stimulating melanocyte proliferation, agents stimulating keratinocyte differentiation, agents stimulating adipocyte differentiation, agents that inhibit acetylcholinesterase, skin relaxant agents, glycosaminoglycan synthesis-stimulating agents, antihyperkeratosis agents, comedolytic agents, antipsoriasis agents, DNA repair agents, DNA protecting agents, stabilizers, anti-itching agents, agents for the treatment and/or care of sensitive skin, firming agents, anti-stretch mark agents, binding agents, agents regulating sebum production, lipolytic agents or agents stimulating lipolysis, anti-cellulite agents, antiperspirant agents, agents stimulating healing, coadjuvant healing agents, agents stimulating reepithelialization, coadjuvant reepithelialization agents, cytokine growth factors, calming agents, anti-inflammatory agents, anesthetic agents, agents acting on capillary circulation and/or microcirculation, agents stimulating angiogenesis, agents that inhibit vascular permeability, venotonic agents, agents acting on cell metabolism, agents to improve dermal-epidermal junction, agents inducing hair growth, hair growth inhibiting or retardant agents, preservatives, perfumes, chelating agents, vegetable extracts, essential oils, marine extracts, agents obtained from a biofermentation process, mineral salts, cell extracts and sunscreens (organic or mineral photoprotective agents active against ultraviolet A and/or B rays) among others, provided they are physically and chemically compatible with the other components of the composition and especially with the peptides of general formula (I) contained in the composition of this invention. Furthermore, the nature of these additional ingredients should not unacceptably alter the benefits of the peptides of this invention. The nature of these additional ingredients can be synthetic or natural, such as vegetable extracts, or obtained by a biofermentation process. Additional examples can be found in the CTFA International Cosmetic Ingredient Dictionary & Handbook, 12th Edition (2008).

An additional aspect of this invention relates to a cosmetic or pharmaceutical composition containing a cosmetically or pharmaceutically effective amount of at least one peptide of the invention according to the general formula (I), its stereoisomers, mixtures thereof and/or its cosmetically or pharmaceutically acceptable salts, and also a cosmetically or pharmaceutically effective amount of at least one extract which is a pigment, a cAMP synthesis stimulating agent, a melanin synthesis stimulating agent, a propigmenting agent, a self-tanning agent and/or an agent stimulating melanocyte proliferation such as, and not restricted to, extracts of Citrus Aurantium Dulcis Fruit, Coleus forskohlii, Coleus Esquirolii, Coleus Scutellariodes, Coleus Xanthanthus, Ballota nigra, Ballota lanata, Ballota suave/ens, Marrubium cylleneum, Cistus creticus, Amphiachyris amoena, Aster oharai, Otostegia fruticosa, Plectranthus barbatus, Halimium viscosum or Larix laricema among others, or at least a synthetic compound or bio-fermentation product which is a pigment, a cAMP synthesis stimulating agent, a melanin synthesis stimulating agent, a propigmenting agent, a self-tanning agent and/or an agent stimulating melanocyte proliferation such as and not restricted to, dihydroxyacetone and derivatives, sugars such as, for example and not restricted to, erythrulose, melanin and its derivatives including melanin polymers and water-soluble low molecular weight melanin derivatives, forskolin and its derivatives including deacetylforskolin and isoforskolin, tyrosine and its derivatives including acetyl tyrosine, oleoyl tyrosine, 3-aminotyrosine and 3-nitrotyrosine, copper salts such as CuCl₂, carotenoids, canthaxanthins, dihydroxyindole carboxylic acid polymers, 3,4-dihydroxybenzoic acid, 3-amino-4-hydroxybenzoic acid, aloin, emodin, alizarin, dihydroxyphenylalanine, 4,5-dihydroxynaphthalene-2-sulphonic acid, 3-dimethylaminophenol or 4-aminobenzoic acid, Heliostatine IS™ [INCI: Pisum Sativum Extract] marketed by Vincience/ISP, Vegetan [INCI: Dihydroxyacetone] or Vegetan Premium [INCI: Dihydroxyacetone, Melanin] marketed by Soliance, MelanoBronze [INCI: Vitex Agnus Castus Extract, Acetyl Tyrosine] marketed by Mibelle Biochemistry, Melitane® [INCI: Acetyl Hexapeptide-1] marketed by Institut Europeen de Biologie Cellulaire/Unipex Innovations, Actibronze® [INCI: Hydrolyzed Wheat Protein, Acetyl Tyrosine, Copper Gluconate] or Instabronze® [INCI: Dihydroxyacetone, Tyrosine] marketed by Alban Muller, Thalitan [INCI: Hydrolyzed Algin, Magnesium Sulfate, Manganese Sulfate] marketed by CODIF, Tyrosilane® [INCI: Methylsilanol Acetyltyrosine] marketed by Exsymol, Tyr-Excel™ [INCI: Oleoyl Tyrosine, Luffa Cylindrica Seed Oil, Oleic Acid] or Tyr-Ol [INCI: Oleoyl Tyrosine, Butylene glycol, Oleic Acid] marketed by Sederma/Croda, Bronzing S.F. [proposed INCI: Butiryl Pentapeptide] marketed by Infinitec Activos or Biotanning® [INCI: Hydrolyzed Citrus Aurantium Dulcis Fruit Extract] marketed by Silab, among others.

An additional aspect of this invention relates to a cosmetic or pharmaceutical composition containing a cosmetically or pharmaceutically effective amount of at least one peptide according to the general formula (I), its stereoisomers, mixtures thereof and/or its cosmetically or pharmaceutically acceptable salts, and also a cosmetically or pharmaceutically effective amount of at least one extract which is an anti-wrinkle agent, antiaging agent such as and not restricted to the extracts of Vitis vinifera, Rosa canina, Curcuma Tonga, Iris pallida, Theobroma cacao, Ginkgo biloba, Leontopodium Alpinum or Dunaliella salina among others or, in addition, at least one synthetic compound or bio-fermentation product which is an anti-wrinkle agent and/or an antiaging agent such as and not restricted to Matrixyl® [INCI: Palmitoyl Pentapeptide-4], Matrixyl 3000® [INCI: Palmitoyl Tetrapeptide-7, Palmitoyl Oligopeptide], Essenskin™ [INCI: calcium hydroxymethionine], Renovage [INCI: teprenone] or Dermaxyl® [INCI: Palmitoyl Oligopeptide] marketed by Sederma/Croda, Vialox® [INCI: Locust Bean (Ceratonia Siliqua) Gum] or Preregen® [INCI: Glycine Soya (Soybean) Protein, Oxido Reductases] marketed by Pentapharm/DSM, Myoxinol™ [INCI: Hydrolyzed Hibiscus Esculentus Extract], Syniorage™ [INCI: Acetyl Tetrapeptide-11], Dermican™ [INCI: Acetyl Tetrapeptide-9] or DN-AGE™ LS [INCI: Cassia Alata leaf Extract] marketed by Laboratoires Serobiologiques/Cognis, Algisum C® [INCI: Methylsilanol Mannuronate] or Hydroxyprolisilane CN® [INCI: Methylsilanol Hydroxyproline Aspartate] marketed by Exsymol, Argireline® [INCI: Acetyl Hexapeptide-8], SNAP-7 [INCI: Acetyl Heptapeptide-4], SNAP-8 [INCI: Acetyl Octapeptide-3], Leuphasyl® [INCI: Pentapeptide-18], Aldenine® [INCI: Hydrolyzed wheat protein, hydrolyzed soy protein, Tripeptide-1], Preventhelia™ [INCI: Tetrapeptide Diaminopropionoyl Tripeptide-33], Trylagen™ [INCI: Pseudoalteromonas Ferment Extract, Hydrolyzed Wheat Protein, Hydrolyzed Soy Protein, Tripeptide-10 Citrulline, Tripeptide-1], Eyeseryl® [INCI: Acetyl Tetrapeptide-5], Peptide AC29 [INCI: Acetyl Tripeptide-30 Citrulline], Lipochroman-6 [INCI: Dimethylmethoxy Chromanol], Chromabright™ [INCI: Dimethylmethoxy Chromanyl Palmitate], Antarcticine® [INCI: Pseudoalteromonas Ferment Extract] Vilastene™ [INCI: Lysine HCl, Lecithin, Tripeptide-10 Citrulline]acetyl-arginyl-phenylglycyl-tryptophyl-phenylglycine, acetyl-arginyl-phenylglycyl-valyl-glycine or acetyl-arginyl-phenylglycyl-valyl-phenylglycine marketed by Lipotec, Kollaren® [INCI: Tripeptide-1, Dextran] marketed by Institut Europeen de Biologie Cellulaire/Unipex Group, Collaxyl® IS [INCI: Hexapeptide-9], Laminixyl IS™ [INCI: Heptapeptide], Orsirtine™ GL [INCI: Oryza Sativa (Rice) Extract], D'Orientine™ IS [INCI: Einkorn (Triticum Monococcum) Extract] or Quintescine™ IS [INCI: Dipeptide-4] marketed by Vincience/ISP, BONT-L-Peptide [INCI: Palmitoyl Hexapeptide-19] marketed by Infinitec Activos, Deepaline™ PVB [INCI: Palmitoyl hydrolyzed Wheat Protein] or Sepilift® DPHP [INCI: Dipalmitoyl Hydroxyproline] marketed by Seppic, Gatuline® Expression [INCI: Acmella oleracea Extract], Gatuline® In-Tense [INCI: Spilanthes Acmella Flower Extract] or Gatuline® Age Defense 2 [INCI: Juglans Regia (Walnut) Seed Extract] marketed by Gattefossé, Thalassine™ [INCI: Algae Extract] marketed by Biotechmarine, ChroNOline™ [INCI: Caprooyl Tetrapeptide-3] or Thymulen-4 [INCI: Acetyl Tetrapeptide-2] marketed by Atrium/Unipex Innovations, EquiStat [INCI: Pyrus Malus Fruit Extract, Glycine Soja Seed Extract] or Juvenesce [INCI: Ethoxydiglycol and Caprylic Triglyceride, Retinol, Ursolic Acid, Phytonadione, Ilomastat] marketed by Coletica, Ameliox [INCI: Carnosine, Tocopherol, Silybum Marianum Fruit Extract] or PhytoCellTec Malus Domestica [INCI: Malus Domestica Fruit Cell Culture] marketed by Mibelle Biochemistry, Bioxilift [INCI: Pimpinella Anisum Extract] or SMS Anti-Wrinkle® [INCI: Annona Squamosa Seed Extract] marketed by Silab, antagonists of the Ca²⁺ channel such as and not restricted to, alverine, manganese or magnesium salts, certain secondary or tertiary amines, retinol and its derivatives, idebenone and its derivatives, Coenzyme Q10 and its derivatives, boswellic acid and its derivatives, GHK and its derivatives, carnosine and its derivatives, DNA repair enzymes such as and not restricted to, photolyase, T4 endonuclease V, or chloride channel agonists among others.

An additional aspect of this invention relates to a cosmetic or pharmaceutical composition which comprises a cosmetically or pharmaceutically effective amount of at least one peptide according to the general formula (I), its stereoisomers, mixtures thereof and/or its cosmetically or pharmaceutically acceptable salts, and, in addition, a cosmetically or pharmaceutically effective amount of at least one extract which is an anti-cellulite agent, lipolytic agent and/or venotonic agent such as and not restricted to, the extracts or hydrolyzed extracts of Bupleurum Chinensis, Cecropia Obtusifolia, Celosia Cristata, Centella Asiatica, Chenopodium Quinoa, Chrysanthellum Indicum, Citrus Aurantium Amara, Coffea Arabica, Coleus Forskohlii, Commiphora Myrrha, Crithmum Maritimum, Eugenia Caryophyllus, Ginkgo Biloba, Hedera Helix (ivy extract), Hibiscus Sabdariffa, Ilex Paraguariensis, Laminaria Digitata, Nelumbium Speciosum, Paullinia Cupana, Peumus Boldus, Phyllacantha Fibrosa, Prunella Vulgaris, Prunus Amygdalus Dulcis, Ruscus Aculeatus (extract of Butcher's Broom) Sambucus Nigra, Spirulina Platensis Algae, Uncaria Tomentosa or Verbena Officinalis among others or at least one synthetic compound, extract or bio-fermentation product which is an anti-cellulite agent, lipolytic agent and/or venotonic agent such as and not restricted to, dihydromyricetin, coenzyme A, lipase, glaucine, aesculin, visnadine, Regu®-Shape [INCI: Isomerized Linoleic Acid, Lecithin, Glycerin, Polysorbate 80] marketed by Pentapharm/DSM, UCPeptide™ V [INCI: Pentapeptide] or AT Peptide™ IS [INCI: Tripeptide-3] marketed by Vincience/ISP, Adiposlim [INCI: Sorbitan Laurate, Lauroyl Proline] marketed by SEPPIC, caffeine, carnitine, escin and/or triethanolamine iodide, among others.

### Applications

Another aspect of this invention relates to the use of at least one of the peptides of general formula (I), its stereoisomers, mixtures thereof and/or its cosmetically or pharmaceutically acceptable salts in the preparation of a cosmetic or pharmaceutical composition for the treatment and/or care of skin and/or hair.

In addition, another aspect of this invention relates to the use of at least one of the peptides of general formula (I), its stereoisomers, mixtures thereof and/or its cosmetically or pharmaceutically acceptable salts in the preparation of a cosmetic or pharmaceutical composition for the treatment and/or care of those conditions, disorders and/or diseases of the skin and/or hair requiring cAMP synthesis stimulation.

Furthermore, this invention relates to the use of at least one of the peptides of general formula (I), its stereoisomers, mixtures thereof and/or its cosmetically or pharmaceutically acceptable salts in the preparation of a cosmetic or pharmaceutical composition for the treatment and/or care of skin and/or hair which stimulates melanin synthesis in the skin and/or hair.

According to another preferred embodiment, this invention relates to the use of at least one of the peptides of general formula (I), its stereoisomers, mixtures thereof and/or its cosmetically or pharmaceutically acceptable salts in the preparation of a cosmetic or pharmaceutical composition for the treatment and/or care of skin and/or hair, which accelerates, intensifies and/or prolongs the skin's tan.

According to a preferred embodiment, this invention relates to the use of a peptide of formula (I), its stereoisomers, mixtures thereof and/or its cosmetically or pharmaceutically acceptable salts in the preparation of a cosmetic or pharmaceutical composition for the treatment and/or care of skin and/or hair which reduces the irregularities of pigmentation, preferably irregularities caused by vitiligo.

According to a preferred embodiment, this invention relates to the use of a peptide of formula (I), its stereoisomers, mixtures thereof and/or their cosmetically or pharmaceutically acceptable salts in the preparation of a cosmetic or pharmaceutical composition for the treatment and/or care of the skin and/or hair which reduces, delays and/or prevents the damage induced by UV radiation.

According to a preferred embodiment, this invention relates to the use of a peptide of formula (I), its stereoisomers, mixtures thereof and/or its cosmetically or pharmaceutically acceptable salts in the preparation of a cosmetic or pharmaceutical composition for the treatment and/or care of the skin and/or hair which reduces, delays and/or prevents the signs of aging and/or photoaging.

Likewise, this invention relates to the use of at least one of the peptides of formula (I), its stereoisomers, mixtures thereof and/or its cosmetically or pharmaceutically acceptable salts in the preparation of a cosmetic or pharmaceutical composition for the treatment and/or care of the skin and/or hair which stimulates lipolysis.

According to a preferred embodiment, this invention refers to the use of a peptide of formula (I), its stereoisomers, mixtures thereof and/or its cosmetically or pharmaceutically acceptable salts in the preparation of a cosmetic or pharmaceutical composition for the treatment and/or care of the skin and/or hair which reduces, delays and/or prevents cellulite.

Examples of cosmetic or pharmaceutical compositions for the treatment and/or care of the skin and/or hair include creams, multiple emulsions such as and not restricted to, oil and/or silicone in water emulsions, water in oil and/or silicone emulsions, water/oil/water or water/silicone/water type emulsions and oil/water/oil or silicone/water/silicone type emulsions, anhydrous compositions, aqueous dispersions, oils, milks, balsams, foams, lotions, gels, cream gels, hydroalcoholic solutions, hydroglycolic solutions, liniments, sera, soaps, serums, polysaccharide films, ointments, mousses, pomades, powders, bars, pencils and sprays or aerosols (sprays), including leave-on and rinse-off formulations, wipes, hydrogels, adhesive patches, non-adhesive patches, microelectric patches or face masks, make-up products such as make-up foundation, for example fluid foundation and compact foundation, make-up removal lotions, make-up removal milks, under-eye concealers, eye shadows, lipsticks, lip protectors, lip gloss and powders, among others.

The compositions containing the peptides of this invention, their stereoisomers, mixtures thereof and/or their cosmetically or pharmaceutically acceptable salts can be applied to the skin and/or hair or can be administered orally or parenterally as necessary to treat and/or care for a condition, disorder and/or disease.

The cosmetic or pharmaceutical compositions concerned in this invention can be applied to the skin by iontophoresis, sonophoresis, electroporation, microelectric patches, mechanical pressure, osmotic pressure gradient, occlusive cure, microinjections or needle-free injections by means of pressure, such as injections by oxygen pressure, or any combination thereof, to achieve a greater penetration of the peptide of the invention.

An additional aspect of this invention relates to a cosmetic or pharmaceutical method for the treatment and/or care of those conditions, disorders and/or diseases of mammals, preferably humans, which require stimulation of cAMP synthesis; which comprises administering an effective amount of at least one peptide of general formula (I), its stereoisomers, mixtures thereof and/or its cosmetically or pharmaceutically acceptable salts, preferably in the form of a cosmetic or a pharmaceutical composition containing them. This invention also provides a cosmetic or pharmaceutical method for stimulating melanin synthesis in the skin and/or hair. Furthermore, this invention provides a cosmetic or pharmaceutical method for accelerating, intensifying and/or prolonging the skin's tan. An additional aspect of this invention relates to a cosmetic or pharmaceutical method for reducing pigmentation irregularities, preferably irregularities caused by vitiligo. Moreover, this invention provides a cosmetic or pharmaceutical method to reduce, delay and/or prevent damage induced by UV radiation. Furthermore, this invention provides a cosmetic or pharmaceutical method to reduce, delay and/or prevent the signs of aging and/or photoaging. This invention also provides a cosmetic or pharmaceutical method for stimulating lipolysis in the skin. Moreover, this invention provides a cosmetic or pharmaceutical method to reduce, delay and/or prevent cellulite.

This invention also provides a cosmetic or pharmaceutical method for the treatment and/or care of those conditions, disorders and/or diseases of the skin and/or hair requiring stimulation of cAMP synthesis, which comprises the topical or transdermic application onto the skin and/or hair or oral or parental administration of a cosmetic or pharmaceutical composition containing at least one peptide of the invention, its stereoisomers, mixtures thereof and/or its cosmetic or pharmaceutical acceptable salts.

The frequency of application or administration can vary greatly, depending on the needs of each subject, with a recommendation of an application or administration range from once a month to ten times a day, preferably from once a week to four times a day, more preferably from three times a week to three times a day, even more preferably once or twice a day.

The following specific examples provided here illustrate the nature of this invention. These examples are included for illustrative purposes only and should not be construed as limitations on the invention claimed herein.

### EXAMPLES

### General Methodology

All reagents and solvents are of synthesis quality and are used without additional treatment.

### Abbreviations

The abbreviations used for amino acids follow the IUPAC-IUB Joint Commission on Biochemical Nomenclature rules outlined in Eur. J. Biochem. (1984) 138:9-37 and in J. Biol. Chem. (1989) 264:633-673.

®, resin; AC, adenylyl cyclase; Ac, acetyl; ACTH, adrenocorticotropic hormone; DNA, deoxyribonucleic acid; Adpoc, 1-(1-adamantyl)-1-methylethoxy-carbonyl; All, allyl; Alloc, allyloxycarbonyl; AM, 2-[4-aminomethyl-(2,4-dimethoxyphenyl)]phenoxyacetic acid; ATP, adenosine triphosphate; Boc, tert-butyloxycarbonyl; 2-BrZ, 2-bromobenzyloxycarbonyl; Bzl, benzyl; cAMP, cyclic adenosine monophosphate; Cbz, carboxybenzyl; cGMP, cyclic guanosine monophosphate; cHx, cyclohexyl; CITrt-®, 2-chlorotrityl resin; CIZ, 2-chlorobenzyl; cps, centipoise; CRE, cAMP response element; CREB, cAMP response element-binding; C-terminal, carboxy-terminal; DCM, dichloromethane; DCT, dopachrome tautomerase; Dde, N-[1-(4,4-dimethyl-2,6-dioxocyclohex-1-ylidene)ethyl; DHA, dihydroxyacetone; 2,6-diCIZ, 2,6-dichlorobenzyl; DIEA, N,N-diisopropylethylamine; DIPCDI, N,N′-diisopropylcarbodiimide; Dmab, 4-(N-[1-(4,4-dimethyl-2,6-dioxocyclohexylidene)-3-methylbutyl]amino)benzyl; DMF, N,N-dimethylformamide; DNA, deoxyribonucleic acid; DNP, 2,4-dinitrophenol; DOPA, 3,4-dihydroxyphenylalanine; DPPC, dipalmitoylphosphatidylcholine; EDTA, ethylenediaminetetraacetic acid; equiv, equivalent; ESI-MS, electrospray ionization mass spectrometry; Fm, fluorenylmethyl; Fmoc, 9-fluorenylmethyloxycarbonyl; HOAt, 1-hydroxy-7-azabenzotriazole; HOBt, 1-hydroxybenzotriazole; HPLC, high performance liquid chromatography; HSL, hormone-sensitive lipase; IBMX, isobutylmethylxanthine; INCI, International Nomenclature of Cosmetic Ingredients; ITA, individual typological angle; ivDde, 1-(4,4-dimethyl-2,6-dioxocyclohexylidene)-3-methyl-butyl; L, luminance; MBHA, p-methylbenzhydrylamine; MC1Rα, human melanocortin-1 receptor; MeCN, acetonitrile; MeOH, methanol; Met, methionine; MITF, microphthalmia-associated transcription factor; MLV, multilaminar vesicles; MPD, minimal pigmenting dose; α-MSH, melanocyte-stimulating hormone; Mtt, methoxytrityl or methyltrityl; q.s., quantity sufficient; q.s.p., quantity sufficient for; Nle, norleucine; N-terminal, amino-terminal; PAL, 5-(4-aminomethyl-3,5-dimethoxyphenoxy)valeric acid; Palm, palmitoyl; Phe, phenylalanine; PKA, protein kinase A; PKC, protein kinase C; pNZ, p-nitrobenzyloxycarbonyl; tBu, tert-butyl; Teoc, 2-(trimethylsilyl)ethyloxycarbonyl; TFA, trifluoroacetic acid; THF, tetrahydrofuran; TIS, triisopropylsilane; Troc, 2,2,2-trichloroethyloxycarbonyl; TRP-1, tyrosinase-related protein-1; Trt, triphenylmethyl or trityl; Trt, trityl; Tyr, tyrosine; ULV, unilaminar vesicles; UV, ultraviolet; Z, benzyloxycarbonyl.

### Chemical Synthesis

All synthetic processes were carried out in polypropylene syringes fitted with porous polyethylene discs or Pyrex® reactors fitted with porous plates. Solvents and soluble reagents were removed by suction. The Fmoc group was removed with piperidine-DMF (2:8, v/v) (1×1 min, 1×5 min, 5 mL/g resin) [Lloyd-Williams P., Albericio F. and Giralt E. (1997) “Chemical Approaches to the Synthesis of Peptides and Proteins” CRC, Boca Raton, Fla., USA]. Washes between stages of deprotection, coupling, and, again, deprotection, were carried out with DMF (3×1 min) each time using 10 mL solvent/g resin. Coupling reactions were performed with 3 mL solvent/g resin. The control of the couplings was performed by carrying out the ninhydrin test [Kaiser E., Colescott R. L., Bossinger C. D. and Cook P. I. (1970) “Color test for detection of free terminal amino groups in the solid-phase synthesis of peptides” Anal. Biochem. 34:595-598]. All synthetic reactions and washes were carried out at room temperature.

HPLC chromatographic analysis was carried out with Shimadzu equipment (Kyoto, Japan) using a reversed-phase column thermostatized at 30° C. (250×4.0 mm, Kromasil C₈, 5 μm, Akzo Nobel, Sweden). The elution was carried out using a gradient of acetonitrile (+0.07% TFA) in water (+0.1% TFA) at a flow rate of 1 mL/min and detection was carried out at 220 nm.

### Example 1 (Prophetic)

### Obtaining Fmoc-AA₁-AA₂-AA₃-O-2-CITrt-®, Wherein AA₃ is -L-Met- or -L-Nle-; AA₂ is -L-Tyr- or -L-Phe- and AA₁ is -L-Tyr- or -L-Phe-

4.04 g of Fmoc-L-Tyr(tBu)-OH or 3.41 g of Fmoc-L-Phe-OH (8.8 mmol; 1 equiv) dissolved in 55 mL of DCM to which is added 1.3 mL of DIEA (7.6 mmol; 0.86 equiv) are coupled onto the dry 2-chlorotrityl resin (5.5 g; 8.8 mmol). They are stirred for 5 min, after which 2.5 mL of DIEA are added (14.6 mmol; 1.66 equiv). The mixture is allowed to react for 40 min. Remaining chloride groups are blocked by treatment with 4.4 mL of MeOH.

The N-terminal Fmoc group is deprotected as described in the general methods and 8.52 g of Fmoc-L-Phe-OH or 10.11 g of Fmoc-L-Tyr(tBu)-OH (22 mmol, 2.5 equiv) are coupled onto the peptidyl resin in the presence of DIPCDI (3.39 mL, 22 mmol, 2.5 equiv) and HOBt (3.37 g, 22 mmol, 2.5 equiv) using DMF as a solvent for 1 hour. The resin is then washed as described in the general methods and the deprotection treatment of the Fmoc group is repeated to couple 7.77 g of Fmoc-L-Nle-OH or 8.17 g of Fmoc-L-Met-OH (22 mmol; 2.5 equiv) using 3.37 g of HOBt (22 mmol; 2.5 equiv) and 3.39 mL of DIPCDI (22 mmol; 2.5 equiv).

After the synthesis, the peptidyl resins are washed with DCM (5×3 min) and dried by nitrogen stream.

### Example 2

### Obtaining Fmoc-AA₁-AA₂-AA₃-AM-MBHA-®, Wherein AA₃ is -L-Met-; AA₂ is -L-Tyr- and AA₁ is -L-Tyr-

6.85 g of the Fmoc-AM-MBHA resin with a functionalization of 0.73 mmol/g (5 mmol) were treated with piperidine-DMF according to the described general protocol in order to remove the Fmoc group. 9.29 g of Fmoc-L-Met-OH (25 mmol; 5 equiv) were incorporated onto the deprotected resin in the presence of DIPCDI (3.85 mL, 25 mmol; 5 equiv) and HOBt (3.85 g, 25 mmol; 5 equiv) using DMF as a solvent for 1 hour.

The resin was then washed as described in the general methods and the deprotection treatment of the Fmoc group was repeated to couple the next amino acid. Following the previously described protocols 11.49 g of Fmoc-L-Tyr(tBu)-OH (25 mmol; 5 equiv) and subsequently 11.49 g of Fmoc-L-Tyr(tBu)-OH (25 mmol; 5 equiv) were coupled sequentially each coupling in the presence of 3.85 g of HOBt (25 mmol; 5 equiv) and 3.85 mL of DIPCDI (25 mmol; 5 equiv).

After the synthesis, the peptidyl resins were washed with DCM (5×3 min) and dried by nitrogen stream.

Alternatively, the same process could have been applied for 8.84 g of Fmoc-L-Nle-OH (25 mmol; 5 equiv) incorporated onto the deprotected resin and/or 9.69 g of Fmoc-L-Phe-OH (25 mmol; 5 equiv) and/or 9.69 g of Fmoc-L-Phe-OH (25 mmol; 5 equiv) sequentially coupled.

### Example 3

### General Process for Removal of Fmoc N-Terminal Protective Group

The N-terminal Fmoc group of the peptidyl resins obtained in Example 2 was deprotected as described in the general methods (20% piperidine in DMF, 1×5 min+1×20 min). The peptidyl resins were washed with DMF (5×1 min), DCM (4×1 min), diethyl ether (4×1 min) and dried under vacuum. The same process could have been applied to the N-terminal group of the peptidyl resins obtained in prophetic Example 1.

### Example 4

### Process for Introducing the R₁ Palmitoyl Group onto the Peptidyl Resins Obtained in Example 3

2.56 g of palmitic acid (10 mmol; 10 equiv) pre-dissolved in DMF (1 mL) were added onto 1 mmol of the peptidyl resins obtained in Example 3, in the presence of 1.53 g of HOBt (10 mmol; 10 equiv) and 1.54 mL of DIPCDI (10 mmol; 10 equiv). They were allowed to react for 15 hours, after which the resins were washed with THF (5×1 min), DCM (5×1 min), DMF (5×1 min), MeOH (5×1 min), DMF (5×1 min) THF (5×1 min), DMF (5×1 min), DCM (4×1 min), ether (3×1 min), and were dried under vacuum.

### Example 5 (Prophetic)

### Process for Introducing the R₁ Acetyl Group onto the Peptidyl Resins Obtained in Example 3

1 mmol of peptidyl resins obtained in Example 3 is treated with 25 equiv of acetic anhydride in the presence of 25 equiv of DIEA using 5 mL of DMF as a solvent. They are allowed to react for 30 mins, after which the peptidyl resins are washed with DMF (5×1 min), DCM (4×1 min), diethyl ether (4×1 min) and are dried under vacuum.

### Example 6

### Cleavage Process from the Polymeric Support of the Peptidyl Resins Obtained in Example 4

200 mg of the dried peptidyl resins obtained in Example 4 were treated with 5 mL of TFA:TIS:H₂O (90:5:5) for 2 hours at room temperature under stirring. Filtrates were collected onto 50 mL cold diethyl ether, they were filtered through polypropylene syringes fitted with porous polyethylene discs and washed 5 times with 50 mL diethyl ether. The final precipitates were dried under vacuum.

HPLC analysis of the obtained peptides in gradients of MeCN (+0.07% TFA) in H₂O (+0.1% TFA) showed a purity exceeding 80% in all cases. The identity of the peptides obtained was confirmed by ESI-MS. The same process could have been applied to the peptidyl resins obtained in Examples 3 and 5.

### Example 7 (Prophetic)

### Cleavage Process of the Polymeric Support and Functionalization with R₂ Substituted Amine: Obtaining Ac-AA₁-AA₂-AA₃-NH—(CH₂)₁₅—CH₃, Wherein AA₃ is -L-Met- or -L-Nle-; AA₂ is -L-Tyr- or -L-Phe- and AA₁ is -L-Tyr- or -L-Phe-

The peptides Ac-AA₁-AA₂-AA₃-OH with fully protected side chains are obtained by treating 150 mg of the peptidyl resins Ac-AA₁AA₂-AA₃-O-2-CITrt-® of Example 5, previously desiccated under vacuum in the presence of KOH, with 3 mL of a 3% solution of TFA in DCM for 5 min. The filtrates are collected onto 50 mL of cold diethyl ether and the treatment is repeated three times. Ethereal solutions are evaporated to dryness at reduced pressure and room temperature, the precipitates are redissolved in 50% MeCN in H₂O and lyophilized. 10 mg of the obtained crude peptides are weighed in a flask and 3 equiv of hexadecylamine and 25 mL of anhydrous DMF are added. 2 equiv of DIPCDI are added, and left to react being magnetically stirred at 47° C. The reactions are monitored by HPLC until disappearance of the initial products, which are complete after 24-48 hours. Solvents are evaporated to dryness and co-evaporated twice with DCM. The obtained residues [Ac-AA₁-AA₂-AA₃-NH—(CH₂)₁₅—CH₃ with fully protected side chains] are redissolved in 25 mL of a mixture of TFA-DCM-anisole (49:49:2) and left to react for 30 min at room temperature. 250 mL of cold diethyl ether are added, the solvents are evaporated under reduced pressure and two additional co-evaporations with ether are carried out. The residues are dissolved in a mixture of 50% MeCN in H₂O and lyophilized.

### Example 8

### cAMP Synthesis Stimulation Assay

cAMP synthesis stimulation was assessed in the human G361 melanocyte cell line in the presence of the peptides of the invention. The cells were seeded (10⁶ cells/plate 25 cm²) and incubated for 24 hours in McCoy's complete medium, after which the peptides were added to 10 μM and were incubated for another 24 hours. 40 μM forskolin was used as a positive control. The cells were centrifuged and the supernatants were collected, and the cAMP levels were determined by carrying out a competitive ELISA assay following the protocols of the commercial kit (Cayman, Ref. 581001)

Table 2 provides details of the peptides which showed cAMP stimulation level values greater than 20%. cAMP levels were normalized with regards to the average basal cAMP values.

TABLE 2
Increase in cAMP levels

| Treatment | cAMP increase |
|---|---|
| Forskolin | 117% |
| Palm-L-Tyr-L-Tyr-L-Met-NH₂ | 57% |

### Example 9

### Melanogenesis Stimulation by Palm-L-Tyr-L-Tyr-L-Met-NH₂

A human G361 melanocyte cell line was incubated for 4 days on a 12-well plate in presence of the peptide at various concentrations, after which the cells were trypsinized, the melanin was extracted and was quantified by measuring the absorbance at 470 nm in a spectrophotometer. The values obtained were normalized with regards to the number of cells. The concentration of melanin was determined in pg/cell using a standard regression analysis obtained with synthetic melanin at known concentrations.

Table 3 shows the melanin synthesis stimulation values obtained by using treatments with Palm-L-Tyr-L-Tyr-L-Met-NH₂ at the study concentrations.

TABLE 3
Melanin synthesis stimulation

| Treatment | Melanin synthesis stimulation |
|---|---|
| Palm-L-Tyr-L-Tyr-L-Met-NH₂ 10 μM | 64% |
| Palm-L-Tyr-L-Tyr-L-Met-NH₂ 50 μM | 79% |
| Palm-L-Tyr-L-Tyr-L-Met-NH₂ 100 μM | 138% |

### Example 10

### Preparation of a Cosmetic Composition Containing Palm-L-Tyr-L-Tyr-L-Met-NH₂

| | | % IN |
|---|---|---|
| INGREDIENT (INCI Nomenclature) | | WEIGHT |
| A | WATER (AQUA) | q.s.p. 100 |
| | PRESERVATIVES | 0.45 |
| | IMIDAZOLIDINYL UREA | 0.095 |
| | DISODIUM EDTA | 0.14 |
| | GLYCERIN | 4.75 |
| | PROPYLENE GLYCOL | 2.85 |
| B | WATER (AQUA), POLYACRYLAMIDE, | 2.85 |
| | C13-14 ISOPARAFFIN, LAURETH-7 | |
| | ETHYLHEXYL COCOATE | 4.75 |
| | CAPRYLIC/CAPRIC TRIGLYCERIDE | 4.75 |
| C | DIMETHICONE | 1.9 |
| D | TRIETHANOLAMINE | q.s. |
| E | FRAGRANCE (PARFUM) | 0.19 |
| F | Palm-L-Tyr-L-Tyr-L-Met-NH₂ 0.01%, BUTYLENE | 5 |
| | GLYCOL, ALCOHOL DENAT | |

Phase A was dissolved in an appropriate reactor. In another reactor, phase B was mixed and once homogenized slowly added onto phase A under stirring. Then phase C was added under stirring, and subsequently phase F was added at 35° C. The pH was adjusted to 5.5-7.0 with phase D and phase E was added.

### Example 11 (Prophetic)

### Preparation of Liposomes Containing Ac-L-Tyr-L-Tyr-L-Nle-NH₂

| | % IN |
|---|---|
| INGREDIENT (INCI Nomenclature) | WEIGHT |
| PHOSPHATIDYLCHOLINE | 4.0 |
| Ac-L-Tyr-L-Tyr-L-Nle-NH₂ | 0.2 |
| PRESERVATIVES | 0.50 |
| AQUA (WATER) | q.s.p. 100 |

Dipalmitoylphosphatidylcholine (DPPC) is weighed and dissolved in chloroform. The solvent is evaporated under vacuum until obtaining a fine phospholipid layer, and this layer is hydrated under treatment at 55° C. with an aqueous solution of the peptide at the desired concentration (containing Phenonip®), and MLV liposomes are obtained. ULV liposomes are obtained by submerging the MLV liposomes in an ultrasound bath at 55° C. for 8 cycles of 2 mins at intervals of 5 mins. The size of the ULV liposomes is reduced by passing them through a high pressure extrusion system.

### Example 12 (Prophetic)

### Preparation of a Composition in the Form of a Liposome Gel Containing Ac-L-Tyr-L-Tyr-L-Nle-NH₂

The liposomes of Example 11 are dispersed in water with the preservatives (EDTA, imidazolidinyl urea and Phenonip®) under light stirring. Hispagel® 200 is added [INCI: Aqua (Water), glycerin, glyceryl polyacrylate] and is lightly stirred until a homogenous mixture is obtained.

| | % IN |
|---|---|
| INGREDIENT (INCI Nomenclature) | WEIGHT |
| LIPOSOMES CONTAINING Ac-L-Tyr-L-Tyr-L-Nle-NH₂ | 10.00 |
| (1%) | |
| DISODIUM EDTA | 0.15 |
| IMIDAZOLIDINYL UREA | 0.10 |
| PRESERVATIVE | 0.50 |
| AQUA (WATER) | 29.25 |
| AQUA (WATER), GLYCERIN, GLYCERYL | 60.00 |
| POLYACRYLATE | |

### Example 13 (Prophetic)

### Composition of a Facial Cream Containing Ac-L-Tyr-L-Phe-L-Met-NH₂

| | % IN |
|---|---|
| INGREDIENT (INCI Nomenclature) | WEIGHT |

| | | |
|---|---|---|
| A | BUTYROSPERMUM PARKII | 3.5-4.5 |
| | CETEARYL ETHYLHEXANOATE | 3-5 |
| | GLYCERYL STEARATE S.E. | 1.5-2.5 |
| | SQUALANE | 0.5-1 |
| | PEG-100 STEARATE | 1 |
| | POLYSORBATE 60 | 0.30 |
| | CETYL PALMITATE | 1.5-2.5 |
| | DIMETHICONE | 2.5-3.5 |
| | CETEARYL ALCOHOL | 1.5-2.5 |
| | PALMITIC ACID | 0.5 |
| B | AQUA (WATER) | 2 |
| | GLYCERIN | 1.5-2.5 |
| | BUTYLENE GLYCOL | 1-3 |
| | MANNITOL | 0.5-1.5 |
| | HYDROGENATED LECITHIN | 0.5-1.5 |
| | PROPYLENE GLYCOL | 0.5-1.5 |
| C | CARBOMER | 0.4 |
| | ETHYLHEXYL PALMITATE | 1.5-2.5 |
| D | TROMETHAMINE | 0.4 |
| | AQUA (WATER) | 1 |
| E | PRESERVATIVES | q.s. |
| F | Ac-L-Tyr-L-Phe-L-Met-NH₂ | 0.001 |
| | AQUA (WATER) | q.s.p.100 |

### Example 14 (Prophetic)

### Preparation of a Composition of Mixed Micelles Containing Ac-L-Phe-L-Tyr-L-Met-OH

The ingredients of phase A are weighed and warmed slightly to about 30° C. to help to dissolve some of the preservatives in a vessel suitable for the complete sample. Next, phase B components are added and homogenized under light stirring.

Phase C is then added under continuous stirring, after which phase D is added with slow stirring to avoid foaming.

The pH is adjusted to 5.5-6.5.

| | | % IN |
|---|---|---|
| INGREDIENT (INCI Nomenclature) | | WEIGHT |
| A | AQUA (WATER) | q.s.p.100 |
| | PHENOXYETHANOL | 0.5 |
| | CAPRILYL GLYCOL | 0.5 |
| | POTASSIUM SORBATE | 0.3 |
| B | AQUA (WATER) | 27.5 |
| | Ac-L-Phe-L-Tyr-L-Met-OH | 0.025 |
| | LECITHIN | 4.0 |
| C | XANTHAN GUM | 0.4 |
| D | AQUA (WATER), CAPRILYL/CAPRYL GLUCOSIDE | 30 |

### Example 15 (Prophetic)

### Preparation of a Microemulsion Composition Containing Palm-L-Tyr-L-Tyr-L-Met-NH₂

| | % IN |
|---|---|
| INGREDIENT (INCI Nomenclature) | WEIGHT |
| DIETHYLHEXYL SODIUM SULFOSUCCINATE | 1.35 |
| ISOSTEARIC ACID | 7.65 |
| AQUA (WATER) | 0.2 |
| ALCOHOL DENAT | 0.8 |
| ETHYLHEXYL COCOATE | 90 |
| Palm-L-Tyr-L-Tyr-L-Met-NH₂ | 0.005 |

### Example 16 (Prophetic)

### Composition of a Capillary Lotion Containing Ac-L-Tyr-L-Tyr-L-Met-OH

| | | % IN |
|---|---|---|
| | INGREDIENT (INCI Nomenclature) | WEIGHT |
| A | ALCOHOL DENAT. | 50-60 |
| | PANTHENOL | 0.05-0.15 |
| | ZINC RICINOLEATE | 0.05-0.10 |
| | FRAGRANCE | 0.02 |
| | Ac-L-Tyr-L-Tyr-L-Met-OH | 0.01 |
| B | AQUA (WATER) | q.s.p.100 |

Phase A components are mixed slowly and under stirring. Phase B is slowly added onto phase A under stirring until fully homogenized.

### Example 17

### Effect of the Composition of Example 10 on the Acceleration, Intensification and Prolonging of Tan

Caucasian volunteers, between 25 and 35 years of age, phototypes II, III, IV (according to Fitzpatrick) applied the cream from Example 10 on their forearm, once a day for 4 weeks and a placebo cream on their other forearm. Both forearms were exposed to UVA irradiation, three times a week for the first two weeks, under controlled conditions. The UVA dosage was chosen between 8 and 25 J/cm² based on the individual MPD (Minimal Pigmenting Dose) and the source of light was positioned directly in contact with the subject's forearm skin. The colorimetry of the forearm skin was assessed instrumentally at the beginning and during the irradiation (7 days) and two weeks after the last irradiation (28 days after beginning the treatment) using the chromameter CR-400.

An increase in the reduction of the ITA values of 109% and luminance of 58% was obtained after 7 days of treatment under UV induction with regards to the placebo, showing an acceleration of skin tanning.

Twenty-eight days after starting the treatment and 14 days after the last UVA irradiation, the areas treated with the cream containing the peptide showed a reduction in luminance of 48% and ITA of 40% with regards to the placebo. These results show that the treatment intensifies and prolongs the skin's tan.

1. A cosmetic or pharmaceutical composition which comprises:
a cosmetically or pharmaceutically effective amount of at least one peptide of general formula (I)
R₁-AA₁-AA₂-AA₃-R₂ (I)
its stereoisomers, mixtures thereof and/or its cosmetic or pharmaceutical acceptable salts, wherein:
AA₁ and AA₂ are independently selected from the group consisting of -Tyr- and -Phe-;
AA₃ is selected from the group consisting of -Nle- and -Met-;
R₁ is selected from the group consisting of H, unsubstituted non-cyclic aliphatic groups, substituted non-cyclic aliphatic groups selected from the group consisting of acetyl, tert-butanoyl, hexanoyl, 2-methylhexanoyl, octanoyl, decanoyl, lauroyl, myristoyl, palmitoyl, stearoyl, oleoyl and linoleoyl, substituted or unsubstituted alicyclyl, substituted or unsubstituted heterocyclyl, substituted or unsubstituted heteroarylalkyl, substituted or unsubstituted aryl, substituted or unsubstituted aralkyl and R₅—CO—; and
R₂ is selected from the group consisting of —NR₃R₄, —OR₃ and —SR₃;
wherein R₃ and R₄ are independently selected from the group consisting of H, unsubstituted non-cyclic aliphatic groups, substituted or unsubstituted alicyclyl, substituted or unsubstituted heterocyclyl, substituted or unsubstituted heteroarylalkyl, substituted or unsubstituted aryl, and substituted or unsubstituted aralkyl; and
wherein R₅ is selected from the group consisting of H, unsubstituted non-cyclic aliphatic groups, substituted or unsubstituted alicyclyl, substituted or unsubstituted aryl, substituted or unsubstituted aralkyl, substituted or unsubstituted heterocyclyl and substituted or unsubstituted heteroarylalkyl; and
at least one cosmetically or pharmaceutically acceptable excipient or adjuvant.

2. The composition according to claim 1, R₁ is R₅CO— and wherein R₅ is selected from the group consisting of unsubstituted C₁-C₂₄ alkyl, unsubstituted C₂-C₂₄ alkenyl, unsubstituted C₂-C₂₄ alkynyl, substituted or unsubstituted C₃-C₂₄ cycloalkyl, substituted or unsubstituted C₅-C₂₄ cycloalkenyl, substituted or unsubstituted C₅-C₂₄ cycloalkynyl, substituted or unsubstituted C₆-C₃₀ aryl, substituted or unsubstituted C₇-C₂₄ aralkyl, substituted or unsubstituted heterocyclyl with 3-10 ring members, and substituted or unsubstituted heteroarylalkyl of 2 to 24 carbon atoms and 1 to 3 atoms other than carbon and an alkyl chain of 1 to 6 carbon atoms.

3. (canceled)

4. The composition according to claim 1, wherein R₂ is —NR₃R₄ or —OR₃, wherein R₃ and R₄ are independently selected from the group consisting of H, unsubstituted C₁-C₂₄alkyl, unsubstituted C₂-C₂₄ alkenyl, unsubstituted C₂-C₂₄ alkynyl, substituted or unsubstituted C₃-C₂₄ cycloalkyl, substituted or unsubstituted C₅-C₂₄ cycloalkenyl, substituted or unsubstituted C₅-C₂₄ cycloalkynyl, substituted or unsubstituted C₆-C₃₀ aryl, substituted or unsubstituted C₇-C₂₄ aralkyl, substituted or unsubstituted heterocyclyl with 3-10 ring members, and substituted or unsubstituted heteroarylalkyl of 2 to 24 carbon atoms and 1 to 3 atoms other than carbon and an alkyl chain of 1 to 6 carbon atoms.

5. The composition according to claim 4, wherein R₃ and R₄ are independently selected from the group consisting of H, methyl, ethyl, hexyl, dodecyl and hexadecyl.

6. The composition according to claim 1, wherein R₁ is selected from the group consisting of acetyl, lauroyl, myristoyl and palmitoyl, AA₁ is -L-Tyr-, AA₂ is -L-Tyr-, AA₃ is -L-Met-, and R₂ is —NR₃R₄ or —OR₃ wherein R₃ and R₄ are independently selected from H, methyl, ethyl, hexyl, dodecyl and hexadecyl.

7. The composition according to claim 1, wherein R₁ is selected from the group consisting of acetyl, lauroyl, myristoyl and palmitoyl, AA₁ is -L-Tyr-, AA₂ is -L-Phe-, AA₃ is -L-Met-, and R₂ is —NR₃R₄ or —OR₃ wherein R₃ and R₄ are independently selected from H, methyl, ethyl, hexyl, dodecyl and hexadecyl.

8. The composition according to claim 1, wherein R₁ is selected from the group consisting of acetyl, lauroyl, myristoyl and palmitoyl, AA₁ is -L-Tyr-, AA₂ is -L-Tyr-, AA₃ is -L-Nle-, and R₂ is —NR₃R₄ or —OR₃ wherein R₃ and R₄ are independently selected from H, methyl, ethyl, hexyl, dodecyl and hexadecyl.

9-22. (canceled)

23. A process for preparation of a composition which comprises a peptide of general formula (I), its stereoisomers, mixtures thereof and/or its cosmetically or pharmaceutically acceptable salts, according to claim 1, comprising synthesizing the peptide in solid phase or in solution and combining the peptide with the at least one cosmetically or pharmaceutically acceptable excipient or adjuvant.

24. (canceled)

25. (canceled)

26. The composition according to claim 25, wherein the peptide of general formula (I) is present in a concentration between 0.000001% and 20% in weight, with regards to the total weight of the composition.

27. (canceled)

28. The composition according to claim 1, wherein the peptide of general formula (I), its stereoisomers, mixtures thereof and/or its cosmetically or pharmaceutically acceptable salts, is incorporated into a cosmetic or pharmaceutical delivery system and/or sustained release system selected from the group consisting of liposomes, mixed liposomes, oleosomes, niosomes, millicapsules, microcapsules, nanocapsules, nanostructured lipid carriers, sponges, cyclodextrins, vesicles, micelles, mixed micelles of surfactants, surfactant-phospholipid mixed micelles, millispheres, microspheres, nanospheres, lipospheres, microemulsions, nanoemulsions, miniparticles, milliparticles, microparticles, nanoparticles and solid lipid nanoparticles or is adsorbed on a cosmetically or pharmaceutically acceptable solid organic polymer or solid mineral support selected from the group consisting of talc, bentonite, silica, starch and maltodextrin.

29.-30. (canceled)

31. The composition according to claim 1, wherein the composition is presented in a formulation selected from the group consisting of creams, multiple emulsions, anhydrous compositions, aqueous dispersions, oils, milks, balsams, foams, lotions, gels, cream gels, hydroalcoholic solutions, hydroglycolic solutions, hydrogels, liniments, sera, soaps, shampoos, conditioners, serums, ointments, mousses, pomades, powders, bars, pencils, sprays, aerosols, capsules, gelatin capsules, tablets, sugar coated tablets, granules, chewing gum, solutions, suspensions, emulsions, syrups, polysaccharide films, jellies and gelatins.

32. The composition according to claim 1, wherein the peptide of general formula (I), its stereoisomers, mixtures thereof and/or its cosmetically or pharmaceutically acceptable salts, is incorporated into a product selected from the group consisting of under-eye concealers, make-up foundation, make-up removing lotions, make-up removing milks, eye shadows, lipsticks, lip gloss, lip protectors and powders.

33. The composition according to claim 1, wherein the peptide of general formula (I), its stereoisomers, mixtures thereof and/or its cosmetically or pharmaceutically acceptable salts, is incorporated into a fabric, a non-woven fabric or a medical device.

34. (canceled)

35. The composition according to claim 1, wherein the at least one adjuvant is selected from the group consisting of other cyclic adenosine monophosphate synthesis stimulating agents, elastase inhibitory agents, matrix metalloproteinase inhibitory agents, melanin synthesis stimulating or inhibiting agents, whitening or depigmenting agents, propigmenting agents, self-tanning agents, antiaging agents, NO-synthase inhibiting agents, 5α-reductase inhibiting agents, lysyl- and/or prolyl hydroxylase inhibiting agents, antioxidants, free radical scavengers and/or agents against atmospheric pollution, reactive carbonyl species scavengers, anti-glycation agents, antihistamine agents, antiemetic agents, antiviral agents, antiparasitic agents, emulsifiers, emollients, organic solvents, liquid propellants, skin and/or hair conditioners, humectants, substances that retain moisture, alpha hydroxyacids, beta hydroxyacids, moisturizers, epidermal hydrolytic enzymes, vitamins, pigments or colorants, dyes, gelling polymers, thickeners, surfactants, softening agents, anti-wrinkle agents, agents able to reduce or treat the bags under the eyes, exfoliating agents, antimicrobial agents, antifungal agents, fungistatic agents, bactericidal agents, bacteriostatic agents, agents stimulating the synthesis of dermal or epidermal macromolecules and/or capable of inhibiting or preventing their degradation, collagen synthesis-stimulating agents, elastin synthesis-stimulation agents, decorin synthesis-stimulation agents, laminin synthesis-stimulation agents, defensin synthesis-stimulating agents, chaperone synthesis-stimulating agents, aquaporin synthesis-stimulating agents, hyaluronic acid synthesis-stimulating agents, fibronectin synthesis-stimulating agents, sirtuin synthesis-stimulating agents, agents stimulating the synthesis of lipids and components of the stratum corneum, agents stimulating the synthesis of ceramides, agents that inhibit collagen degradation, agents that inhibit elastin degradation, agents that inhibit serine proteases such cathepsin G, agents stimulating fibroblast proliferation, agents stimulating keratinocyte proliferation, agents stimulating adipocyte proliferation, agents stimulating melanocyte proliferation, agents stimulating keratinocyte differentiation, agents stimulating adipocyte differentiation, agents that inhibit acetylcholinesterase, skin relaxant agents, glycosaminoglycan synthesis-stimulating agents, antihyperkeratosis agents, comedolytic agents, antipsoriasis agents, DNA repair agents, DNA protecting agents, stabilizers, anti-itching agents, agents for the treatment and/or care of sensitive skin, firming agents, anti-stretch mark agents, binding agents, agents regulating sebum production, lipolytic agents or agents stimulating lipolysis, anti-cellulite agents, antiperspirant agents, agents stimulating healing, coadjuvant healing agents, agents stimulating reepithelialization, coadjuvant reepithelialization agents, cytokine growth factors, calming agents, anti-inflammatory agents, anesthetic agents, agents acting on capillary circulation and/or microcirculation, agents stimulating angiogenesis, agents that inhibit vascular permeability, venotonic agents, agents acting on cell metabolism, agents to improve dermal-epidermal junction, agents inducing hair growth, hair growth inhibiting or retardant agents, preservatives, perfumes, chelating agents, vegetable extracts, essential oils, marine extracts, agents obtained from a biofermentation process, mineral salts, cell extracts and sunscreens, organic or mineral photoprotective agents active against ultraviolet A and/or B rays, and mixtures thereof.

36-42. (canceled)

43. The composition according to claim 1 wherein AA₂ is -Tyr-.

44. The composition according to claim 1, wherein when AA₁ is -Phe-, AA₂ is -Tyr-, AA₃ is -Met-, and R₂ is —NH₂, then R₁ is not acetyl.

45. A cosmetic or pharmaceutical method for the treatment and/or care of the skin and/or hair which comprises administering the composition of claim 1 to the skin and/or hair.

46. The cosmetic or pharmaceutical method according to claim 45 for the treatment and/or care of those conditions, disorders and/or diseases of the skin and/or hair requiring stimulation of cyclic adenosine monophosphate synthesis.

47. The cosmetic or pharmaceutical method according to claim 45 in which the treatment and/or care stimulates melanin synthesis.

48. The cosmetic or pharmaceutical method according to claim 47 in which the treatment and/or care accelerates, intensifies and/or prolongs the skin's tan.

49. The cosmetic or pharmaceutical method according to claim 45 in which the treatment and/or care reduces the pigmentation irregularities of the skin and/or hair, reduces and/or delays damage induced by UV radiation, reduces and/or delays the signs of aging and/or photoaging, stimulates lipolysis, and/or reduces and/or delays cellulite.

50. A cosmetic or pharmaceutical method for the treatment and/or care of the skin and/or hair which comprises administering a composition to the skin and/or hair, the composition comprising an effective amount of at least one peptide of general formula (I),
R₁-AA₁-AA₂-AA₃-R₂ (I)
its stereoisomers, mixtures thereof and/or its cosmetic or pharmaceutical acceptable salts, wherein:
AA₁ and AA₂ are independently selected from amongst themselves from the group consisting of -Tyr- and -Phe-;
AA₃ is selected from the group consisting of -Nle- and -Met-;
R₁ is selected from the group consisting of substituted and unsubstituted non-cyclic aliphatic groups, substituted and unsubstituted alicyclyls, substituted and unsubstituted heterocyclyl groups, substituted and unsubstituted heteroarylalkyl groups, substituted or unsubstituted aryl groups, substituted or unsubstituted aralkyl groups and R₅—CO—; and
R₂ is selected from the group consisting of —NR₃R₄, —OR₃ and —SR₃;
wherein R₃ and R₄ are independently selected from the group consisting of H, substituted and unsubstituted non-cyclic aliphatic groups, substituted and unsubstituted alicyclyl groups, substituted and unsubstituted heterocyclyl groups, substituted and unsubstituted heteroarylalkyl groups, substituted and unsubstituted aryl groups, and substituted and unsubstituted aralkyl groups; and
wherein R₅ is selected from the group consisting of H, substituted or unsubstituted non-cyclic aliphatic group, substituted or unsubstituted alicyclyl, substituted or unsubstituted aryl, substituted or unsubstituted aralkyl, substituted or unsubstituted heterocyclyl and substituted or unsubstituted heteroarylalkyl;
its stereoisomers, mixtures thereof, and/or its cosmetically or pharmaceutically acceptable salts; and
at least one cosmetically or pharmaceutically acceptable excipient or adjuvant.
